(19) United States
(12) Patent Application Publication
(10) Pub. No.: US 2005/0234312 A1
(43) Pub. Date: Oct. 20, 2005

(54) Bio-information measuring apparatus

(75) Inventors: Takuji Suzuki, Kanagawa (JP); Kenichi Kameyama, Kanagawa (JP); Akihisa Moriya, Tokyo (JP); Kazushige Ouchi, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOSHIBA, Tokyo (JP)

(21) Appl. No.: 11/085,577

(22) Filed: Mar. 22, 2005

(30) Foreign Application Priority Data
Mar. 30, 2004 (JP) 2004-101318
Mar. 30, 2004 (JP) 2004-101319
Jan. 6, 2005 (JP) 2005-001850

(51) Int. Cl. A61B 5/00; G09B 3/00

(52) U.S. Cl. 600/300

(57) ABSTRACT

A bio-information measuring includes a detector including a light source emitting light to a hand of an examinee and an optical receiver receiving the light emitted from the light source; and a supporter bent to clamp a paddle between fingers of the examinee. The clamping portion of the supporter is provided with the detector. The bio-information measuring apparatus also includes a measurer worn on the examinee, measuring bio-information based on pulse wave data obtained from the optical receiver; and a puller pulling the support member toward the measurer.

### CROSS-REFERENCE TO RELATED APPLICATIONS

This application is based upon and claims the benefit of priority from Japanese Patent Applications No. 2004-101318 and No. 2004-101319, both filed Mar. 30, 2004; and Japanese Patent Application No. 2005-001850, filed Jan. 6, 2005, the entire contents of both of which are incorporated herein by reference.

### BACKGROUND OF THE INVENTION

1) Field of the Invention

The present invention relates to a bio-information measuring apparatus that serves to measure the bio-information of a user on the basis of a pulse wave between the user's fingers or the like, and particularly, the present invention relates to a configuration of the apparatus and an art to set a condition for performing communication on the basis of the measured bio-information.

2) Description of the Related Art

A pulse wave can be measured by irradiating a finger or an ear lobe of an examinee with light and then detecting reflected light or transmitted light. A pulse wave rate can be calculated from the measured pulse wave. A pulse wave rate calculator is used in for example, a pulse wave monitor, for cardiopulmonary exercise testing.

By irradiating a finger and an ear lobe of an examinee with two light beams different in wavelength, an infrared light and a red light, and then detecting reflected light or transmitted light, it is possible to measure a blood oxygen saturation level in arterial blood. Such an apparatus is used mainly for management of a respiratory state at a medical field as a pulse wave oximeter.

The pulse wave data can be used not only for measurement of the pulse wave rate or the blood oxygen saturation level but also for other purpose, for example, for determination of a sleeping state of the examinee with combined to body motion data showing the body motion of the examinee. In addition, by constantly measuring the blood oxygen saturation level during sleeping by the pulse wave oximeter, screening of a sleep apnea syndrome can be also carried out.

In the view of such a purpose of use, an apparatus that measures the bio-information such as a pulse wave and a blood oxygen saturation level or the like (a bio-information measuring apparatus) is necessarily worn by the examinee for a long time. However, the above-described bio-information measuring apparatus is configured so as to wind a sensor head including a light source and an optical receiver around a finger by a supporter or put it between fingers by a clip, and it is assumed that this apparatus is used for a specific usage. Therefore, it is not assumed that a conventional bio-information measuring apparatus is used for a long time and if the user wears it for a long time, the user has a strong pain. In the medical field, a sensor capable of being worn for a long time is also used, however, wearing of this type is very troublesome because the sensor head is needed to be fixed by an adhesive plaster or the like.

Therefore, an apparatus of a ring type (for example, see Japanese Patent Application Laid-Open Nos. 2001-70264 and 2001-224088) and an apparatus of a belt type for a baby (for example, see Japanese Patent Application Laid-Open No. 2001-224561) to calculate the pulse wave or the blood oxygen saturation level are suggested, which are designed to be attached for a long time. These apparatuses incorporate a light source and a optical receiver at the inside shaped in a ring, calculate the reflected light and the transmitted light, and display if a result is transmitted to the outside wirelessly or display the result on a display of the ring-shaped apparatus.

An art that analyzes fluctuation of a cardiac beat of the examinee by measurement of the pulse wave and an autonomic state is determined from its result has been known (for example, see Japanese Patent Application Laid-Open No. H7-143972). Particularly, it is suggested to measure the autonomic state and a sleeping state when sleeping in real time by using this art so as to control an external appliance such as home electric appliances or the like.

However, if the autonomic state and a sleeping state are transmitted for each time of measurement in the bio-information measuring apparatus, electric power consumption is increased and a battery is remarkably burn. In order for the user to use the apparatus comfortably, the apparatus is needed to be used for a long time by a predetermined battery, and if the bio-information of the user is obtained, it is preferable that power saving of the apparatus is carried out on the basis of the obtained bio-information.

Therefore, an art to carry out power saving of the apparatus on the basis of the bio-information or the environment information of the user who uses the apparatus has been suggested (for example, see Japanese Patent Application Laid-Open No. 2001-100870). For example, this art serves to determine if the user uses an information processor from the bio-information or the environment information of the user, and only when the user does not use the information processor, supply of a power to the information processor is controlled.

However, in the case of the ring type apparatus disclosed in Japanese Patent Application Laid-Open Nos. 2001-70264 and 2002-224088, it is necessary to coincide the sizes of the ring and the finger upon requests to shield the light from the outside, to receive the light emitted from the light source through the same position of the finger by the optical receiver, and to fix the apparatus to the finger or the like. In addition, it is necessary to measure the size of the finger of the examinee in advance upon attaching the apparatus to the examinee and to prepare the apparatus coinciding with the measured finger size. Upon these reasons, the above-described ring type apparatus involves a problem such that the operations from attachment to measurement cannot be easily carried out.

The belt type apparatus disclosed in Japanese Patent Application Laid-Open No. 2001-224561 serves to be attached soffly by the belt, and this intends to prevent damage of a skin of a baby due to the baby's motion and a large compression from being given to one portion of the skin due to growth of the baby but it does not intend to enable to attach the apparatus without requesting the size of a lower leg of the baby. Therefore, it is necessary to prepare the belt type apparatus that fits the size of the lower leg of the baby and is attached to the lower leg of the baby in consideration of the baby's growth, so that this involves a problem such that the operations from attachment to measurement cannot be easily carried out.

The art disclosed in Japanese Patent Application Laid-Open No. H7-143972 intends power saving by controlling supply of the power when the user does not use the information processor, so that this art cannot be used in the bio-information measuring apparatus, which is needed to calculate the bio-information of the user in real time while activating constantly. In addition, the art disclosed in Japanese Patent Application Laid-Open No. 2001-100870 intends power saving by controlling the power within the appliances and it does not intend power saving when communicating the bio-information.

If the exterior appliance such as home electric appliances is controlled on the basis of the bio-information, it is not necessary to transmit the bio-information constantly. When the user is sleeping, it is perceived that the appliances can be sufficiently controlled if the bio-information is transmitted only when it is changed. On the other hand, when the user is awakening, many noises are included in the bio-information when the user is moving, so that it is perceived that the appliances can be sufficiently controlled even if only the bio-information when the user is not moving is transmitted.

### SUMMARY OF THE INVENTION

It is an object of the present invention to at least solve the problems in the conventional technology.

A bio-information measuring apparatus according to one aspect of the present invention includes a detector including a light source emitting light to a hand of an examinee and an optical receiver receiving light transmitted through the hand from the light source; and a supporter bent to clamp a paddle between fingers of the examinee. The clamping portion of the supporter is provided with the detector. The bio-information measuring apparatus also includes a measurer worn on the examinee, measuring bio-information based on pulse wave data obtained from the optical receiver; and a puller pulling the support member toward the measurer.

A bio-information measuring apparatus according to another aspect of the present invention includes a parameter calculator calculating parameter indicating a state of the autonomic nerve activation based on a pulse wave; a body motion measurer measuring body motion information indicating a body motion of an examinee; a sleep/awake determiner determining whether the examinee is awakening or sleeping based on the body motion information; and a body motion determiner determining whether the examinee is moving based on the body motion information when the sleep/awake determiner determines that the examinee is awakening. The bio-information measuring apparatus also includes a first communication interface transmits the parameter calculated by the parameter calculator to an external device via a network when the body motion determiner determines that the examinee is not moving; a sleeping state determiner determining sleeping state information indicating a depth of sleeping from the parameter calculated by the parameter calculator when the sleep state determiner determines that the examinee is sleeping; a state change determiner determines whether the sleeping state information is changed compared with sleeping state information that has been determined by the sleeping state determiner; and a second communication interface transmits the sleeping state information to the external device via the network when the state change determiner determines that the sleeping state information is changed.

A bio-information measuring apparatus according to still another aspect of the present invention includes a parameter calculator calculating parameter indicating a state of the autonomic nerve activation based on a pulse wave; a body motion measurer measuring body motion information indicating a body motion of an examinee; a sleep/awake determiner determining whether the examinee is awakening or sleeping based on the body motion information; and a body motion determiner determining whether the examinee is moving based on the body motion information when the sleep/awake determiner determines that the examinee is awakening. The bio-information measuring apparatus also includes a first communication interface transmits the parameter calculated by the parameter calculator to an external device via a network when the body motion determiner determines that the examinee is not moving; a sleeping body motion determiner determining whether the examinee is moving in sleep based on the body motion information when the sleep/awake determiner determines that the examinee is sleeping; a sleeping state determiner determining sleeping state information indicating a depth of sleeping from the parameter calculated by the parameter calculator when the body motion determiner determines that the examinee is moving; and a second communication interface transmits the sleeping state information to the external device via the network.

The other objects, features, and advantages of the present invention are specifically set forth in or will become apparent from the following detailed description of the invention when read in conjunction with the accompanying drawings.

### BRIEF DESCRIPTION OF THE DRAWINGS

FIG. 1 is a block diagram of a bio-information measuring apparatus according to a first embodiment of the present invention;

FIG. 2 shows a state that an examinee wears the bio-information measuring apparatus according to the first embodiment;

FIG. 3A is a side view of a sensor head of the bio-information measuring apparatus, FIG. 3B is a top view of the sensor head, and FIG. 3C is a front view of the sensorhead;

FIG. 4 shows a state that the sensor head is attached on the hand of the examinee;

FIG. 5 is a cross sectional view of the sensor head and the hand of the examinee at a line of X-X′ shown in FIG. 4.

FIG. 6 is a cross sectional view of a part of the sensor head;

FIG. 7 is a cross sectional view of a part of another sensor head;

FIG. 8 is a cross sectional view of still another sensor head;

FIG. 9 shows a state that a cable of the bio-information measuring apparatus is pulled by a cable winder;

FIG. 10 shows the cable winder that is incorporated in a bio-information processor of the bio-information measuring apparatus;

FIG. 11 shows a slip ring that is incorporated in the bio-information processor of the bio-information measuring apparatus;

FIG. 12 shows another bio-information processor that is provided with another cable winder of a longitudinal winding;

FIG. 13 shows a display example of the blood oxygen saturation level (SpO2) and the pulse wave rate;

FIG. 14 shows a display example of the blood oxygen saturation level lowering frequency;

FIG. 15 shows a display example of oxygen desaturation Index (ODI);

FIG. 16A is a side view of a sensor head in a first modification of the bio-information measuring apparatus, and FIG. 16B is a top view of the sensor head;

FIG. 17A is a side view of a sensor head in a second modification of the bio-information measuring apparatus, and FIG. 17B is a top view of the sensor head;

FIG. 18A is a side view of a sensor head in a third modification of the bio-information measuring apparatus, and FIG. 16B is a top view of the sensor head;

FIG. 19A is a side view of a sensor head in a fourth modification of the bio-information measuring apparatus, and FIG. 19B is a top view of the sensor head;

FIG. 20A is a side view of a sensor head in a fifth modification of the bio-information measuring apparatus, and FIG. 20B is a top view of the sensor head;

FIG. 21A is a side view of a sensor head in a sixth modification of the bio-information measuring apparatus, and FIG. 21B is a top view of the sensor head;

FIG. 22A is a side view of a sensor head in a seventh modification of the bio-information measuring apparatus, and FIG. 22B is a top view of the sensor head;

FIG. 23A is a side view of a sensor head in an eighth modification of the bio-information measuring apparatus, and FIG. 23B is a top view of the sensor head;

FIG. 24A is a side view of a sensor head in a ninth modification of the bio-information measuring apparatus, and FIG. 24B is a top view of the sensor head;

FIG. 25 is a side view of a sensor head in a tenth modification of the bio-information measuring apparatus;

FIG. 26A is a side view of a sensor head in an eleventh modification of the bio-information measuring apparatus, and FIG. 26B is a top view of the sensor head;

FIG. 27A is a side view of a sensor head in a twelfth modification of the bio-information measuring apparatus, and FIG. 27B is a top view of the sensor head;

FIG. 28 shows two cable winders in a thirteenth modification of the bio-information measuring apparatus;

FIG. 29A shows the bio-information measuring apparatus at the back side of the hand in the thirteen modification, and FIG. 29B shows the bio-information measuring apparatus at the palm side in the thirteen modification;

FIG. 30 is a block diagram of a bio-information measuring apparatus according to a second embodiment of the present invention;

FIG. 31 shows a cable of the bio-information measuring apparatus according to the second embodiment;

FIG. 32 shows another cable of the bio-information measuring apparatus according to the second embodiment;

FIG. 33 is a block diagram of a bio-information measuring apparatus according to a third embodiment of the present invention, a communication device communicating with the bio-information measuring apparatus, and a lighting and an air conditioner which are controlled by a PC;

FIG. 34 is a flowchart of processing in the bio-information measuring apparatus according to the third embodiment;

FIG. 35 is a data graph of a body motion rate during awaking and sleeping at an acceleration detected by an acceleration sensor in the bio-information measuring apparatus according to the third embodiment;

FIG. 36 is a data graph of transmission timing of sleeping state data based on sleeping state in the bio-information measuring apparatus according to the third embodiment;

FIG. 37 is a block diagram of a bio-information measuring apparatus according to a fourth embodiment of the present invention, a communication device communicating with the bio-information measuring apparatus, and a lighting and an air conditioner which are controlled by a PC;

FIG. 38 is a flowchart of processing in the bio-information measuring apparatus according to the fourth embodiment; and

FIG. 39 is a data graph of transmission timing of sleeping state data based on body motion in sleep the bio-information measuring apparatus according to the fourth embodiment.

### DETAILED DESCRIPTION

With reference to the attached drawings, the preferred embodiment(s) of the bio-information measuring apparatus according to the present invention will be described in detail below.

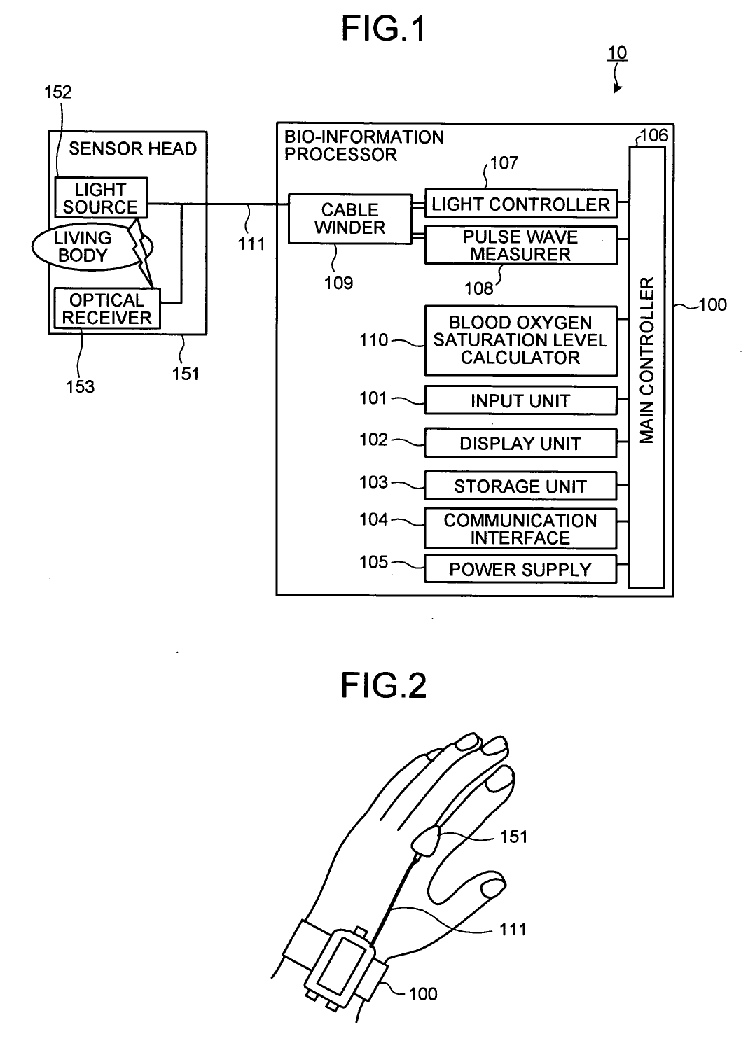

FIG. 1 is a block diagram that depicts a configuration of a bio-information measuring apparatus according to a first embodiment of the present invention. As shown in FIG. 1, the bio-information measuring apparatus 10 has a bio-information processor 100 and a sensor head 151 that is connected to the bio-information processor 100 via a cable 111. In addition, the bio-information measuring apparatus 10 has an input unit 101, a display unit 102, a storage unit 103, a communication interface 104, a power supply 105, a main controller 106, a light controller 107, a pulse wave measurer 108, a cable winder 109, and a blood oxygen saturation level calculator 110.

When the examinee wears the bio-information measuring apparatus 10, it is possible to measure the bio-information of the examinee. FIG. 2 shows a state that the examinee wears the bio-information measuring apparatus 10 according to the first embodiment. In FIG. 2, the examinee puts the sensor head 151 between his or her fingers and the bio-information processor 100 and winds the bio-information processor 100 around his or her wrist. In the meantime, in FIG. 2, the sensor head 151 is attached between the index finger and the long finger, however, the present embodiment is not limited to this and the sensor head 151 may be attached between any fingers.

Returning to FIG. 1, the input unit 101 is used to turn on and off of the power supply by the examinee and switch display of the display unit 102 to be described later, or set a condition necessary for measurement of the bio-information.

The storage unit 103 may store the measurement data such as the pulse wave, the data after calculation processing such as the blood oxygen saturation level data, or a parameter for correction necessary to calculate the blood oxygen saturation level or the like. For example, the storage unit 103 is a flash memory.

The power supply 105 may supply the power to the bio-information processor 100. When the bio-information processor 100 is provided with the power supply 105, it is possible to calculate the pulse wave rate and the blood oxygen saturation level from measurement of the pulse wave with the bio-information measuring apparatus 10 attached. In addition, when the bio-information processor 100 is provided with the power supply 105, it is possible to make the size of the power supply 105 larger than the conventional ring type one, and this makes it possible that the bio-information measuring apparatus 10 can be used for a long time.

The light controller 107 may control intervals of driving of the light source 152 so that the infrared light or the red light is emitted as a pulse wave.

The pulse wave measurer 108 may convert the output current from the sensor head 151 to be described later into a voltage by a current-voltage converter and amplifies the voltage by an amplifier to pass through a high pass filter (a cut off frequency 0.1 Hz) and a low pass filter (a cut off frequency 100 Hz). Then, by converting it into a digital amount by a ten bit A/D converter, the pulse wave measurer 108 may obtain the pulse wave data. The obtained pulse wave data is outputted to the main controller 106. In the meantime, the cut off frequencies of the high pass filter and the low pass filter are not limited to the above-described values.

The main controller 106 may control respective units incorporated in the bio-information measuring apparatus 10 and the output and input of the data. Further, the main controller 106 can detect the transmitted light by an infrared LED and the transmitted light by a red LED selectively by obtaining a signal from a photo diode of an optical receiver 153 in response to a driving timing of the infrared LED or the red LED composing the light source 152. In addition, the main controller 106 can calculate the correction data to deny the influence of the outside light by obtaining a signal of the optical receiver 153 when respective LED are not emitted.

The blood oxygen saturation level calculator 110 may obtain a ratio of a pulse wave component of the infrared LED or the red LED obtained from the pulse wave measurer 108 to calculate the blood oxygen saturation level by using the parameter for correction that is stored in the storage unit 103. In the meantime, as a method to calculate the blood oxygen saturation level, any method is available.

The sensor head 151 is attached between the examinee's fingers to calculate the pulse wave data of the examinee by the light source 152 and the optical receiver 153 that are provided to the sensor head 151. The light source 152 is composed of light emitting diodes of the infrared LED (for example, a wavelength of 940 nm) and the red LED (for example, a wavelength of 660 nm). According to the first embodiment, the light source 152 is arranged at a back of a hand, and the optical receiver 153 is arranged at a palm, however, the present embodiment is not limited to this and the light source 152 may be arranged at the palm and the optical receiver 153 may be arranged at the back of a hand.

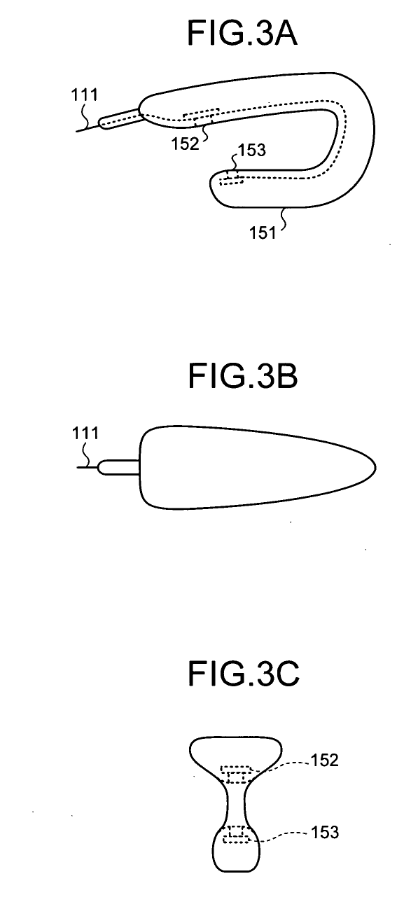

FIGS. 3A to 3C are a shape of the sensor head 151. FIG. 3A is a side view of the sensor head 151, FIG. 3B is a top view thereof, and FIG. 3C is a front view thereof. A main body of the sensor head 151 is made of a solid resin such as epoxy or the like and has the light source 152 and the optical receiver 153 composing inside of the resin and a detecting unit. This resin portion functions as a support member that supports the sensor head 151 between the fingers and has a clamping portion that is bent along a paddle portion between the fingers of the examinee. The sensor head 151 is fixed by a clamping force of this clamping portion between the back of the hand and the palm. Further, it is preferable that the resin forming the sensor head 151 is curved with its opposite sides made concave inwardly so as to be clipped between the fingers without resistance as shown in FIG. 3C, and in addition, as shown in FIG. 3B, it is preferable that the shape of the resin at the back of the hand is made wider in a direction from the bent portion toward the wrist Thereby, it is possible for the user to wear the sensor head 151 very stably, and the examinee can move his or her fingers comfortably although the examinee wears the sensor head 151. In addition, since the main body of the sensor head 151 is made of a resin, an appropriate elasticity can be obtained by the sensor head 151 and a sense of fitting upon wearing the sensor head 151 between the fingers is improved.

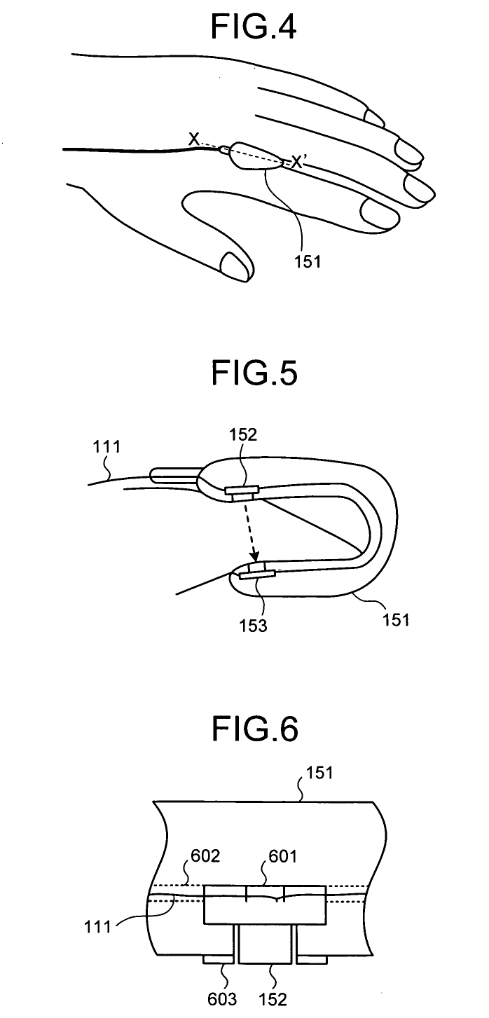

FIG. 4 is a state that the sensor head 151 is attached on the hand of the examinee. In addition, FIG. 5 is a cross sectional view of the sensor head 151 and the hand of the examinee at a line of X-X′ shown in FIG. 4. As shown in FIG. 5, the light emitted from the light source 152 is received by the optical receiver 153 as the transmitted light that is transmitted through the paddle portion of the examinee's hand. By detecting this transmitted light, it becomes possible to measure the bio-information such as the pulse wave and the blood oxygen saturation level or the like.

A method to incorporate the light source 152 and the optical receiver 153 into the sensor head 151 according to the first embodiment will be described below. FIG. 6 is a cross sectional view of the sensor head 151, and particularly, FIG. 6 is a portion where the light source 152 is arranged. A spacer 601 with elasticity is provided between the support member of the sensor head 151 and the light source 152, and the cable 111 that is connected to the light source 152 is guided to the outside of the sensor head 151 through a cable through hole 602. By this spacer 601, the light source 152 is slightly projected from the surface of the support member of the sensor head 151. By this projection, a contacting ability between the light source 152 and the paddle portion of the examinee when wearing the sensor head 151 is improved. In other words, a distance from the light source 152 to the optical receiver 153 is made fixed constantly, and stable measurement of the bio-information can be realized. Further, a cushion 603 is provided so as to surround a periphery of the light source 152, and thereby, it is possible to shield the light source 152 from the outside light In addition, a substrate of the light source 152 is embedded in the sensor head 151, so that the light source 152 is fixed on the sensor head 151 stably.

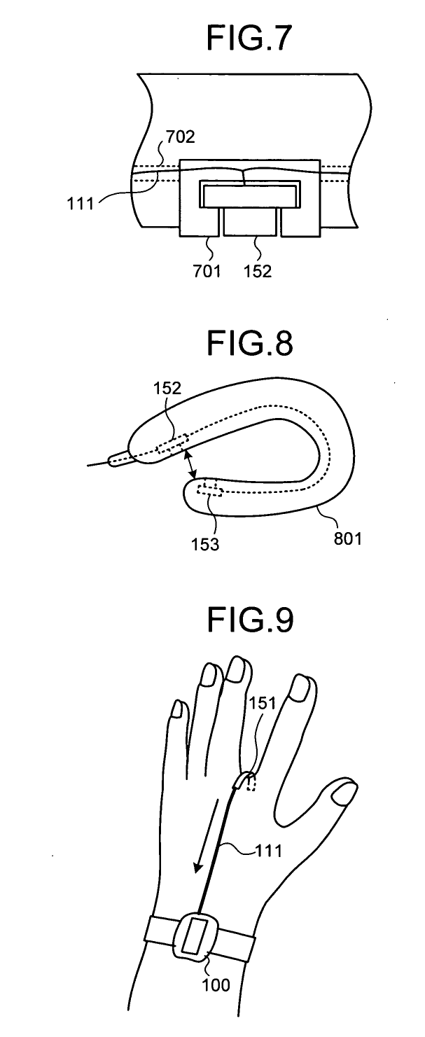

In place of the configuration of the light source 152 shown in FIG. 6, for example, as shown in FIG. 7, a cushion 701 wrapping the light source 152 may be provided in the support member of the sensor head 151. In this case, the cable 111 from the light source 152 is guided to the outside of the sensor head 151 via the cushion 701 through the cable through hole 702. Also in this configuration, as same as FIG. 6, the light source 152 is projected from the sensor head 151, and thereby, a contacting ability between the light source 152 and the paddle portion of the examinee is improved and the outside light can be shielded by the cushion 701. In addition, the cushion 701 wrapping the light source 152 can be fixed in the sensor head 151 by adhesion, so that the light source 152 can be easily attached in the sensor head 151. In the meantime, in FIG. 6 and FIG. 7, the light source 152 is only explained, however, the optical receiver 153 can also be provided in the sensor head 151 by the same configuration.

The support member of the sensor head 151 may be made of other resin other than the solid resin such as epoxy or the like. FIG. 8 shows an example of a sensor head 801 when the support member of the sensor head 151 is made of a silicon rubber. As shown in FIG. 8, according to the support member of a silicone rubber, in order to heighten a contacting ability between the support member and the paddle portion between the fingers, an interval between the clamping portion shown by an arrow is made narrow, and the sensor head 801 is fixed to the paddle portion by the clamping force to be generated by this. The portions other than the clamping portion may be the same as the sensor head 151 shown in FIGS. 3A to 3C. In addition, since the silicon rubber has higher elasticity as compared to the solid resin such as epoxy or the like, the above-described cushion 603 or spacer 601 is unnecessary in the sensor head 801, and a sense of fitting when the examinee wears the sensor head 801 can be improved.

Returning to FIG. 1, the cable 111 may include a signal line for transmitting and receiving a signal to the light source 152 and a signal from the optical receiver 153 to and from the bio-information processor 100 therein. In addition, the cable 111 is pulled by a cable winder 109 with a predetermined tension.

FIG. 9 shows a concept that the cable 111 is pulled by the cable winder 109 that is incorporated in the bio-information processor 100. In order to make the explanation simple, in FIG. 9, the shape of the sensor head 151 is simplified. As shown in FIG. 9, by hitching the sensor head 151 between the fingers of the examinee and pulling the cable 111 by the cable winder 109 by a predetermined tension, despite motion of the hand of the examinee, it is possible to fix the sensor head 151 between the fingers. In the meantime, the predetermined tension can be determined as an appropriate value by actual measurement

Returning to FIG. 1, the cable winder 109 is used to pull the cable 111 by a predetermined tension. In addition, when the examinee does not wear the bio-information measuring apparatus 10, the cable winder 109 is used to put the cable 111 within the bio-information processor 100.

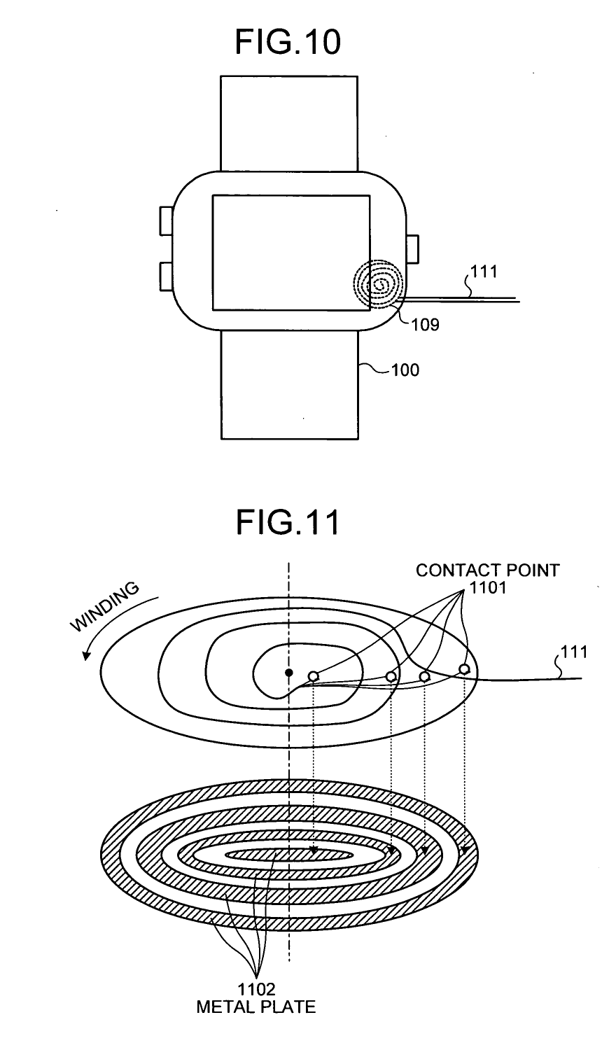

Winding of the cable by the cable winder 109 will be explained below. FIG. 10 shows the cable winder 109 incorporated in the bio-information processor 100 by a dotted line. As shown in FIG. 10, the cable 111 can be winded by a lateral winding. The winding force by this cable winder 109 becomes a predetermined tension for pulling the above-described cable 111.

The cable winder 109 has a rotatable mechanism for winding the cable 111 within the bio-information processor 100. Particularly, a slip ring is used as this rotatable mechanism so that a signal from a signal line in the cable 111 is inputted in the bio-information processor 100. FIG. 11 shows a slip ring that is incorporated in the bio-information processor 100. As shown in FIG. 11, four signal lines in the cable 111 are connected to four contact points 1101. In the meantime, four signal lines are two signal input and output lines, one power source line, and one ground line. In addition, the bio-information processor 100 is provided with four ring-shaped metal plates 1102 corresponding to four contact points 1101, respectively. Respective contact points 1101 contact the metal plates 1102 constantly irrespective of the winding state of the cable 111. Thereby, the bio-information processor 100 can obtain a signal from a signal line in the cable 111 via the metal plate 1102.

The cable winder 109 may be configured so that it can be attached to or detached from the bio-information processor 100. Specifically, it is conceivable that a cartridge forming the sensor head 151, the cable 111, and the cable winder 109 integrally is mounted on the bio-information processor 100 changeably. The cable winder 109 may also be configured so that the cable absorbs torsion caused by the rotation without the slip ring.

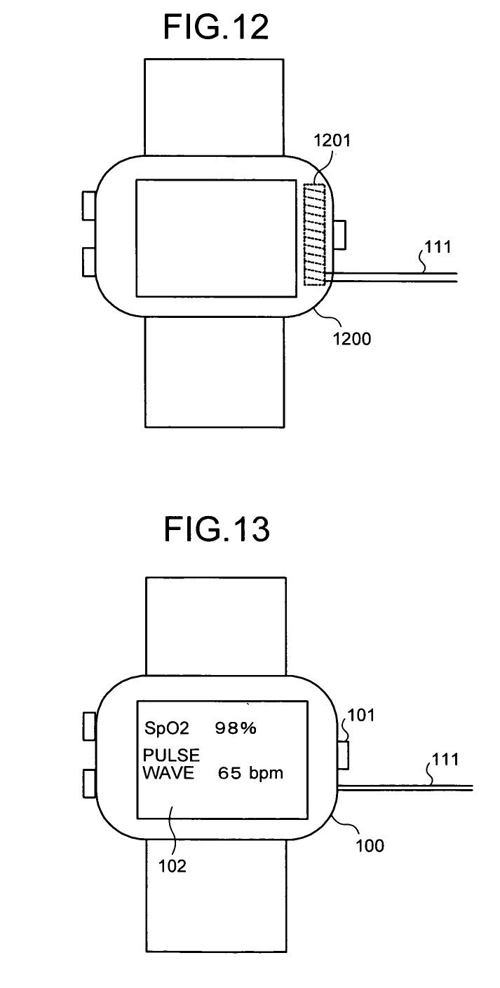

The cable winder 109 may wind the cable 111 by a longitudinal winding not limited to the lateral winding. FIG. 12 shows a bio-information processor 1200 that is provided with a cable winder 1201 of a longitudinal winding. In FIG. 12, the cable winder 1201 is represented by a dotted line.

The display unit 102 may display a pulse wave rate or a calculation result of the blood oxygen saturation level. Specifically, a LCD (Liquid Crystal Display) or the like is conceivable. In addition, since the display unit 102 is provided in the bio-information processor 100 that is separated from the sensor head 151, as compared to the conventional ring-shaped apparatus, it is possible to secure a sufficiently large display area.

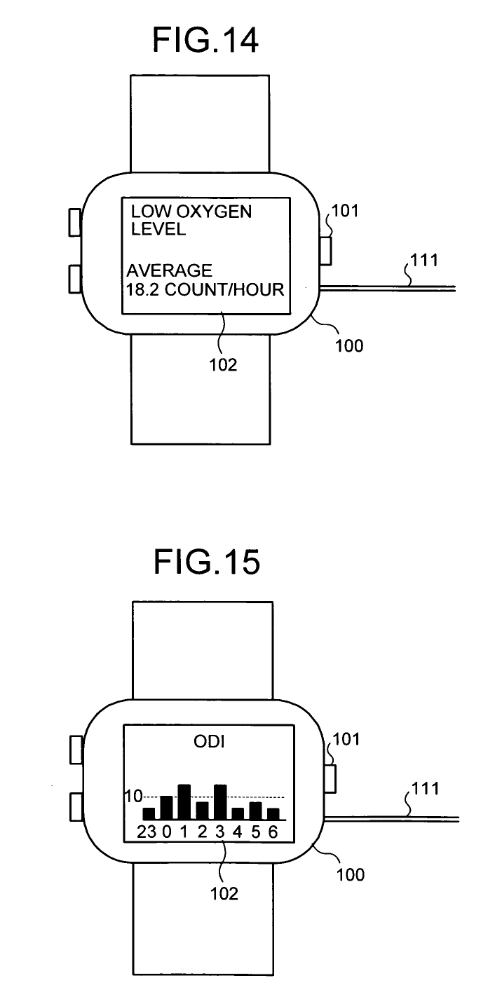

FIG. 13, FIG. 14, and FIG. 15 shows an example that the information measured by the bio-information measuring apparatus 10 is displayed on the display unit 102. Switching of display is carried out by input of the examinee from the input unit 101. FIG. 13 shows a display example of the blood oxygen saturation level (SpO2) and the pulse wave rate. FIG. 14 shows a display example of the blood oxygen saturation level lowering frequency (ODI: Oxygen Desaturation Index). FIG. 15 shows an example that, for example, if 4% and more is lowered from the average of the blood oxygen saturation level, this lowering is counted, and a frequency of this lowering is displayed for each hour on the display unit 102. Thus, by using the bio-information measuring apparatus 10 according to the first embodiment, without having a sense of discomfort due to attachment of the apparatus during sleeping, screening of a sleep apnea syndrome can be carried out.

The communication interface 104 may carry out the data communication with a personal computer and a PDA terminal that manage the measurement result of the bio-information by the wireless communication using a Bluetooth (a registered trademark) and the infrared ray or the wire communication via a communication cable. By transmitting the measurement result to the exterior appliance with the communication interface 104, the measurement result can be preserved in the exterior appliance and viewed by a third party.

As the bio-information measuring apparatus 10 according to the first embodiment, the sensor head 151 and the bio-information processor 100 are formed separately, so that by attaching the sensor head 151 stably, it is possible to measure the bio-information without a pain and a problem in a daily life and to measure the bio-information for a long time by the bio-information processor 100.

The attachment of the apparatus is completed only by attaching the bio-information processor 100 around the wrist and attaching the sensor head 151 between the fingers, so that attachment indifferent to a body type of the examinee becomes possible and a simple attaching method can be provided. In addition, the sensor head 151 formed in accordance with a shape between the fingers is attached and the attached sensor head 151 is pulled by the cable winder 109 by a predetermined tension, so that the light source 152 and the optical receiver 153 are fixed with a high stability and stable measurement of the pulse wave can be realized without having the influence due to the motion and the posture of the examinee. Thereby, the measurement accuracy of the bio-information such as the pulse wave or the blood oxygen saturation level or the like is improved.

In the meantime, according to the first embodiment, a signal line connecting the sensor head 151 and the bio-information processor 100 with each other is included within the cable 111, however, the cable 111 and the signal line are made by different lines and the sensor head and the bio-information processor 100 may be connected with each other by respective lines. In addition, the signal is obtained from the signal line included in the cable 111 not only via the slip ring but also by any method if the signal can be obtained from the signal line.

In the meantime, the present invention is not limited to the above-described first embodiment and various modifications to be described below are available. Further, the drawing seeing the sensor head in each modification from a side is a cross sectional view at the same position as a line X-X′ shown in FIG. 4, in which the sensor head is attached between the index finger and the long finger.

According to the first embodiment, the support member of the sensor head 151 is made of a resin. However, the sensor head 151 is not limited to a shape that can be made of a resin and the sensor head 151 may be formed in a bending shape to clip the paddle portion between the fingers of the examinee with the light source provided at one side of a clamping portion and the optical receiver provided in other side of the clamping portion.

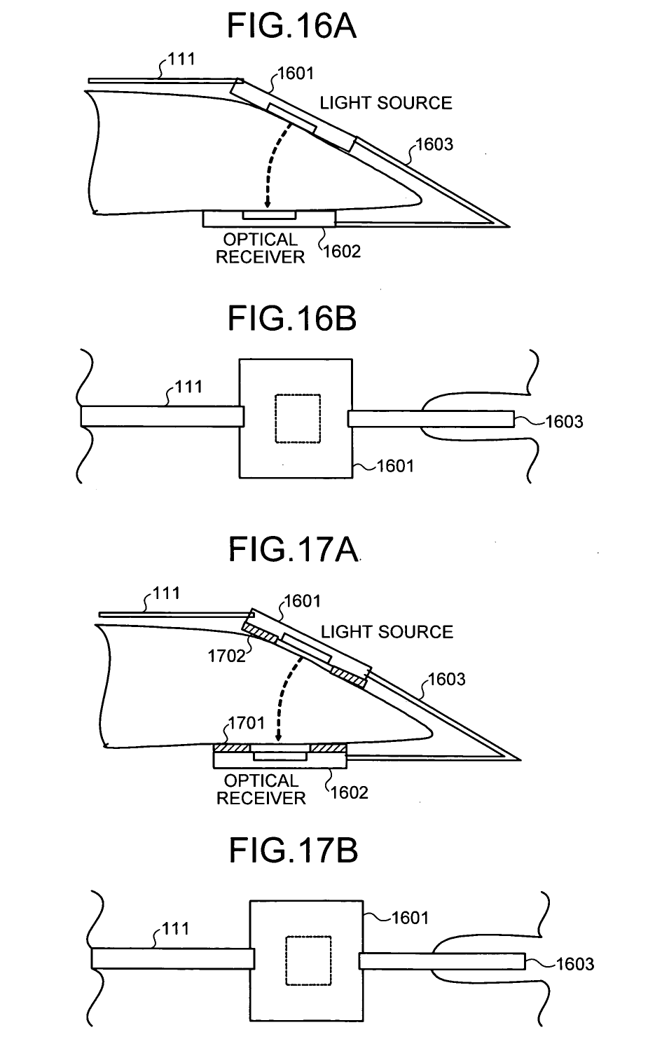

FIG. 16A a side view of a sensor head in a first modification of the bio-information measuring apparatus, and FIG. 16B is a top view of the sensor head. In the first modification, the sensor head is configured by a light source 1601, an optical receiver 1602, and a support portion 1603 of a wedge shape. The support portion 1603 can be made of a material with elasticity enough to be clipped between the fingers, for example, a plastic. The light source 1601 and the optical receiver 1602 configuring the detecting unit are provided at the opposite ends of the support portion 1603. This support portion 1603 is equivalent to the support member of the sensor head. The light source 1601 is connected to the bio-information processor 100 via the cable 111. In addition, the optical receiver 1602 is connected to the bio-information processor 100 via inside of the support portion 1603 and the cable 111. According to this first modification, it is possible to generate the sensor head more economically. In the meantime, in FIG. 16A, the light source 1601 is arranged at the back of the hand and the optical receiver 1602 is arranged at the palm, however, inverse of this is available. This is not limited to the first modification and this is available to a tenth modification to be described later.

FIG. 17A is a side view of a sensor head in a second modification of the bio-information measuring apparatus, and FIG. 17B is a top view of the sensor head. In the second modification, cushions 1702 and 1701 are attached to each of the light source 1601 and the optical receiver 1602 of the sensor head shown in the first modification. As shown in FIG. 17A, a cushion 1702 is provided around the LED in the light source 1601 and the cushion 1701 is provided around the photo diode in the optical receiver 1602. As the cushions 1701 and 1702, a material of a sponge can be used. In addition to the material of a sponge, for example, a patch of a gel type is available. However, it is necessary for the patch of a gel type to be a replaceable shape. In the second modification, by attaching a cushion material, a contacting ability with respect to a surface of a skin of the examinee is improved and the outside light can be shielded.

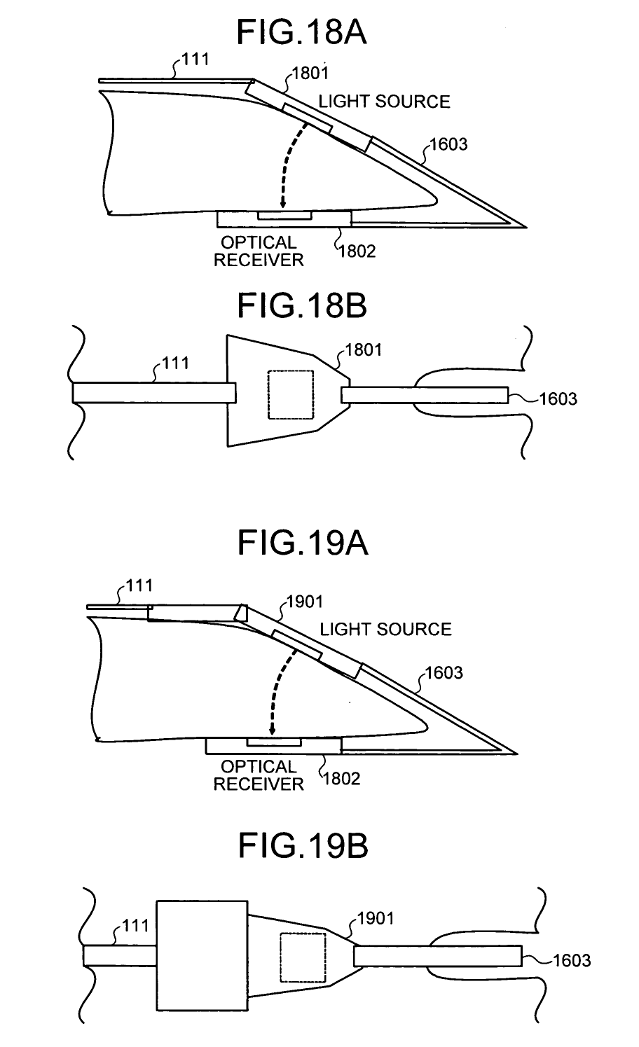

FIG. 18A is a side view of a sensor head in a third modification of the bio-information measuring apparatus, and FIG. 16B is a top view of the sensor head. In the third modification, the support portion 1603 as same as the manner shown by the first modification is used, however, in a light source 1801 and a optical receiver 1802, its width is made narrower the vicinity of the support portion 1603 and is made wider far from the support portion 1603. In the third modification, by providing the light source 1801 and the optical receiver 1802 as shown in FIG. 18B, a sense of discomfort between the fingers upon attachment is decreased and a stable measurement can be realized while decreasing the rotations in a wrist direction and in a vertical direction.

FIG. 19A is a side view of a sensor head in a fourth modification of the bio-information measuring apparatus, and FIG. 19B is a top view of the sensor head. In the fourth modification, in place of the light source 1801 at the back of the hand indicated in the third modification, a light source 1901 is used, which is longer in a direction of the cable 111 and is formed as a plane while being bent in the middle. As shown in FIG. 19A, the light source 1901 at the back of the hand is bent in the middle at the side of the support portion 1603 and at the side of the wrist so as to follow the back of the hand, and the light source 1901 at the wrist is adjusted so as to be angled in parallel with the optical receiver 1802 at the back of the hand. According to this fourth modification, further, the bio-information can be stably measured despite motion of the hand.

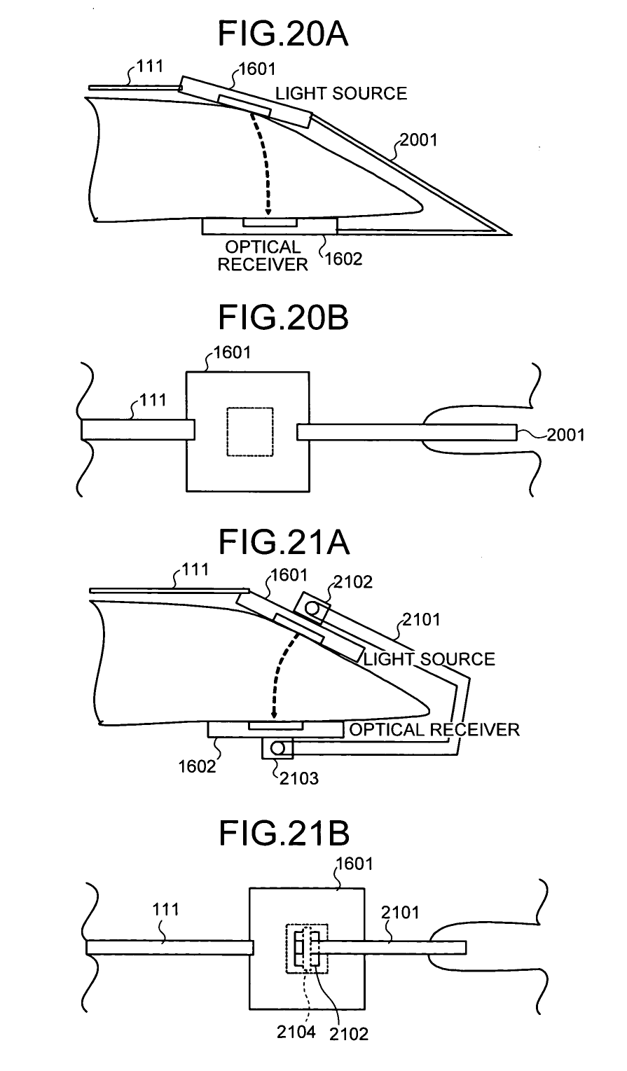

FIG. 20A is a side view of a sensor head in a fifth modification of the bio-information measuring apparatus, and FIG. 20B is a top view of the sensor head. In the fifth modification, in place of the support portion 1603 indicated in FIG. 16A, a support portion 2001 is used. As compared to the support portion 1603, in the support portion 2001, the length from a contacting point with the light source 1601 at the back of the hand to its bent front end is longer than the length from a contacting point with the optical receiver 1602 at the palm to its front end. As shown in FIG. 20A, since the light source 1601 is arranged further near to the wrist than the paddle portion, the examinee can close the fingers without being interrupted by the light source 1601. Thereby, stability in arrangement of the light source 1601 is increased and a sense of fitting of the examinee is improved. However, according to the fifth modification, a thickness of a portion through which the light emitted from the light source 1601 is transmitted is higher than that of the paddle portion, so that it is necessary to make the strength of the light emitted from the light source 1601 stronger than that of the above-described first embodiment and the first to fourth modifications enough to be received at the optical receiver 1602.

FIG. 21A is a side view of a sensor head in a sixth modification of the bio-information measuring apparatus, and FIG. 21B is a top view of the sensor head. In the sixth modification, rotating members 2102 and 2103 are provided between the light source 1601 or the optical receiver 1602 and the support portion 2101. For example, as shown in FIG. 21B, the rotating members 2102 and 2103 are configured by a rotating axis 2104 that is coupled to a point of the support portion 2101, and thereby, the support portion 2101 is slidably coupled to the light source 1601 or the optical receiver 1602. By these rotating members 2102 and 2103, the light source 1601 and the optical receiver 1602 constantly contact the surface of the paddle portion of the examinee despite motion of the hand of the examinee. This means that the influences of the outside light and the influences of the motion of the examinee in detection are decreased, and the stable measurement of the bio-information can be made. In the meantime, the rotating members 2102 and 2103 are shown in FIG. 21B as a mechanism including the rotating axis 2104 rotating only in one direction, however, a member whereby the light source 1601 and the optical receiver 1602 can rotate in plural directions may be available. For example, if a spherical joint is used as the rotating members 2102 and 2103, freedom of the rotating axis can be obtained and a sense of fitting of the sensor head and stability of measurement of the bio-information can be more improved.

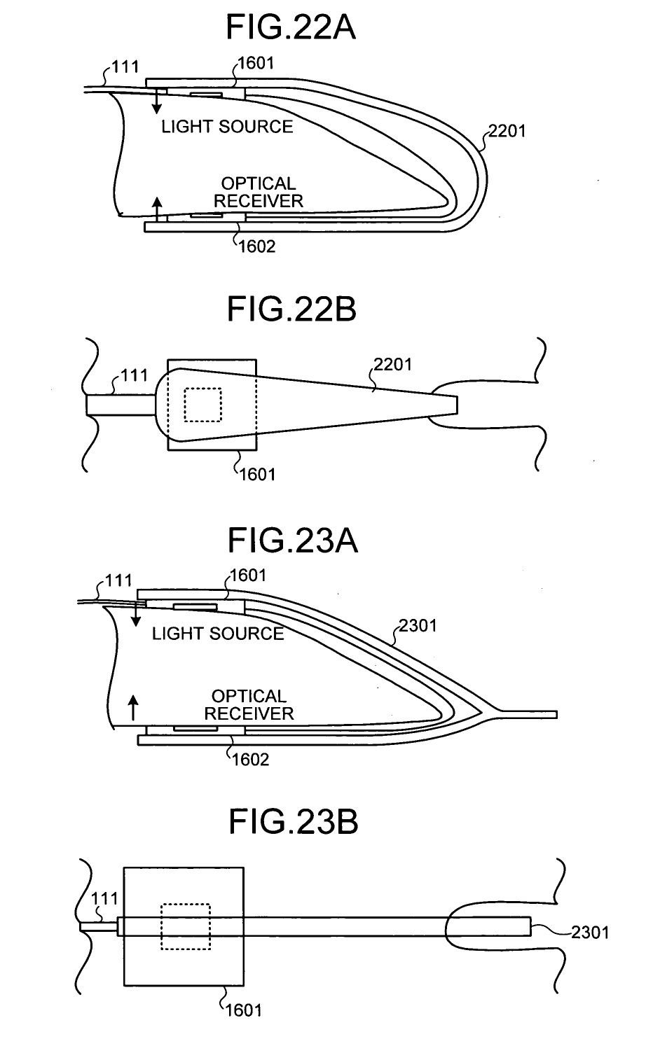

FIG. 22A is a side view of a sensor head in a seventh modification of the bio-information measuring apparatus, and FIG. 22B is a top view of the sensor head. In the seventh modification, a plate spring 2201 formed by bending a metal plate is used as a support member of the sensor head, and the light source 1601 and the optical receiver 1602 are provided on the inner surface of this bent plate spring 2201. As shown in FIG. 22A, the signal line guided from the optical receiver 1602 in the cable 111 via the light source 1601 is provided at the inside of the plate spring 2201. According to this seventh modification, the paddle portion between the fingers of the examinee is pressed by a predetermined pressure so as to prevent the sensor head from being misaligned. In addition, since a contacting ability between the light source 1601 and the paddle portion and the optical receiver 1602 and the paddle portion are improved, the outside light is shielded and a distance from the light source 1601 to the optical receiver 1602 is made constant. Thereby, more stable measurement of the bio-information can be made.

FIG. 23A is a side view of a sensor head in an eighth modification of the bio-information measuring apparatus, and FIG. 23B is a top view of the sensor head. In the eighth modification, a plate spring 2301 formed as a pin set mechanism by putting metal plates together is used as a support member of the sensor head, and the light source 1601 and the optical receiver 1602 are provided on the inner surface of the plate spring 2301. According to this eighth modification, as same as the seventh modification, the paddle portion between the fingers of the examinee is pressed by a predetermined pressure so as to prevent the sensor head from being misaligned. In addition, the outside light is shielded and a distance from the light source 1601 to the optical receiver 1602 is made constant. Thereby, stable measurement of the bio-information can be made.

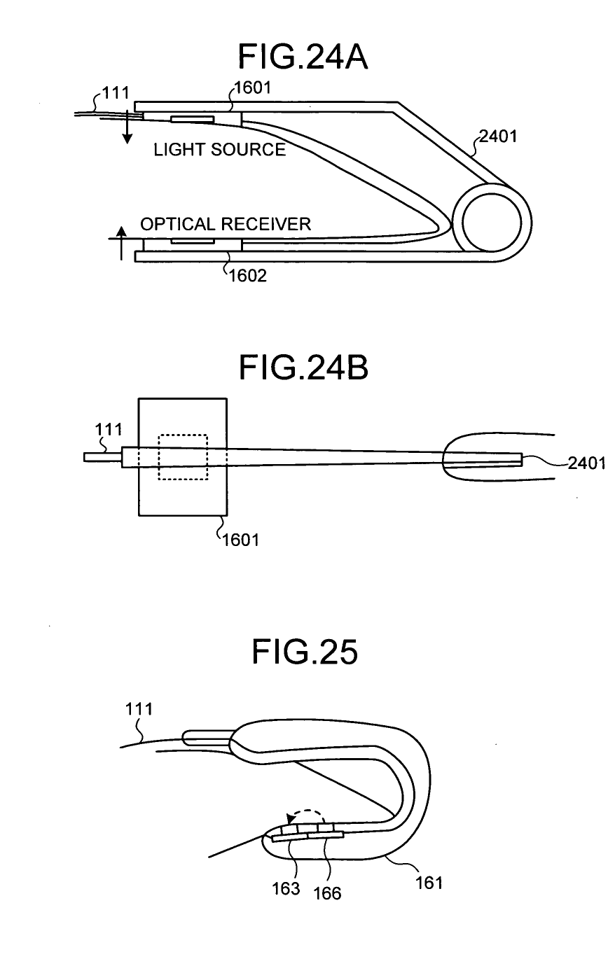

FIG. 24A is a side view of a sensor head in a ninth modification of the bio-information measuring apparatus, and FIG. 24B is a top view of the sensor head. In the ninth modification, a support member of the sensor head is formed by a piano wire of a diameter about 1 mm or a clip spring 2401 made of a stainless steel or the like, and the light source 1601 and the optical receiver 1602 are provided on the inner surface of the clip spring 2401. According to this ninth modification, as same as the seventh modification, the paddle portion between the fingers of the examinee is pressed by a predetermined pressure so as to prevent the sensor head from being misaligned. In addition, the outside light is shielded and a distance from the light source 1601 to the optical receiver 1602 is made constant. Thereby, stable measurement of the bio-information can be made.

According to the first to ninth modifications of the first embodiment, a so-called transmission type of a detecting unit that is provided with a light source at one side of the clamping portion of the sensor head 151 and an optical receiver at other side thereof is provided. However, in place of this, a so-called reflection type of a detecting unit may be available, in which both of the light source and the optical receiver are provided at one side of the clamping portion of the sensor head. FIG. 25 is a side view of a sensor head 161 that is provided with a reflection type of a detecting unit as a tenth modification, and particularly, a side view corresponding to FIG. 3A is illustrated. As shown in FIG. 25, according to the tenth modification, a light source 162 and an optical receiver 163 are arranged adjacently at the palm in the resin forming the support member of the sensor head 161. The light emitted from the light source 162 is reflected in the paddle portion to enter the optical receiver 163. Thereby, the sensor head 161 can transmit the pulse wave data to the bio-information processor 100 via the cable 111. In the meantime, in FIG. 25, the light source 162 and the optical receiver 163 are arranged along a longitudinal direction of the support member of the sensor head 161 (a direction from the bent portion toward the wrist), however, not limited to this arranging direction, the light source 162 and the optical receiver 163 may be arranged vertically to the longitudinal direction of the support member of the sensor head 161.

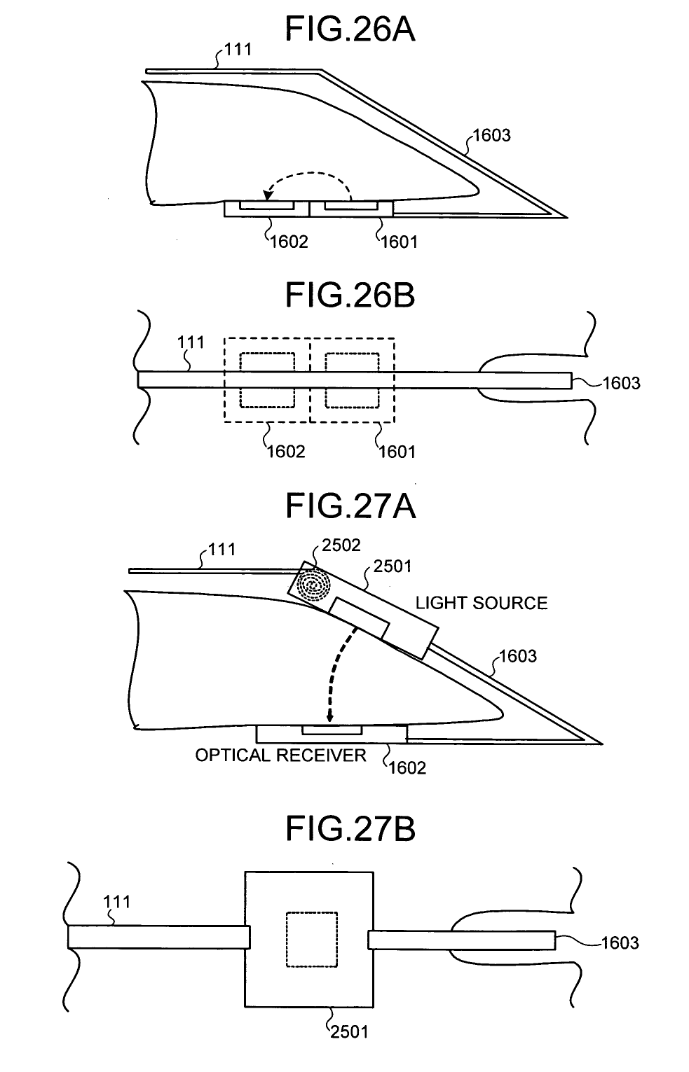

FIG. 26A is a side view of a sensor head in an eleventh modification of the bio-information measuring apparatus, and FIG. 26B is a top view of the sensor head. In the eleventh modification, in the configuration indicated in the first modification, in place of the detecting unit of the transmission type, the above-described detecting unit of the transmission type is provided. As shown in FIG. 26A and FIG. 26B, both of the light source 1601 and the optical receiver 1602 are provided at the palm of the support portion 1603. Particularly, in these drawings, the light source 1601 and the optical receiver 1602 are arranged from the front end of the bent portion toward the wrist. However, not limited to this arranging direction, the light source 1601 and the optical receiver 1602 may be arranged vertically to the longitudinal direction of the support portion 1603. In addition, as same as this modification 11, in other second to ninth modifications, the detecting unit of the reflection type can be employed, and also in this case, the advantages according to the above-described respective modifications can be enjoyed.

FIG. 27A is a side view of a sensor head in a twelfth modification of the bio-information measuring apparatus, and FIG. 27B is a top view of the sensor head. According to the above-described first embodiment and modifications, the cable winder 109 is incorporated in the bio-information processor 100, however, according to the twelfth modification, a cable winder 2502 is incorporated in the sensor head. Specifically, as shown in FIG. 27A, the cable winder 2502 is incorporated in a light source 2501. In the meantime, FIG. 27A and FIG. 27B are a structure having a cable winder in the sensor head configured as indicated in the first modification, however, the present twelfth modification can be applied to the second to eleventh modifications in the same way. Thereby, the bio-information processor 100 is not necessarily provided with the cable winder 109 and if a connector to be connected to the sensor head is provided, it is possible to measure the bio-information such as the blood oxygen saturation level.

According to the first embodiment and modifications, the sensor head is connected to the bio-information processor 100 by one cable 111 from the back of the hand, however the present embodiment is not limited to this. For example, the sensor head attached between the fingers may be connected to the bio-information processor 100 via a cable extended from the back of the hand. In addition, the connection from the sensor head to the bio-information processor 100 is not limited to the connection by one cable and the sensor head may be connected to the bio-information processor 100 by plural cables.

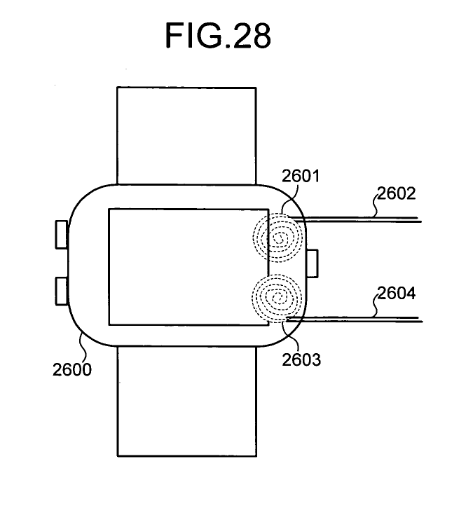

FIG. 28 shows two cable winders 2601 and 2603 are provided within a bio-information processor 2600 that is equivalent to the bio-information processor 100 indicated in the first embodiment Cables 2602 and 2604 are guided from the bio-information processor 2600 to the outside, which are connected to the cable winders 2601 and 2603, respectively.

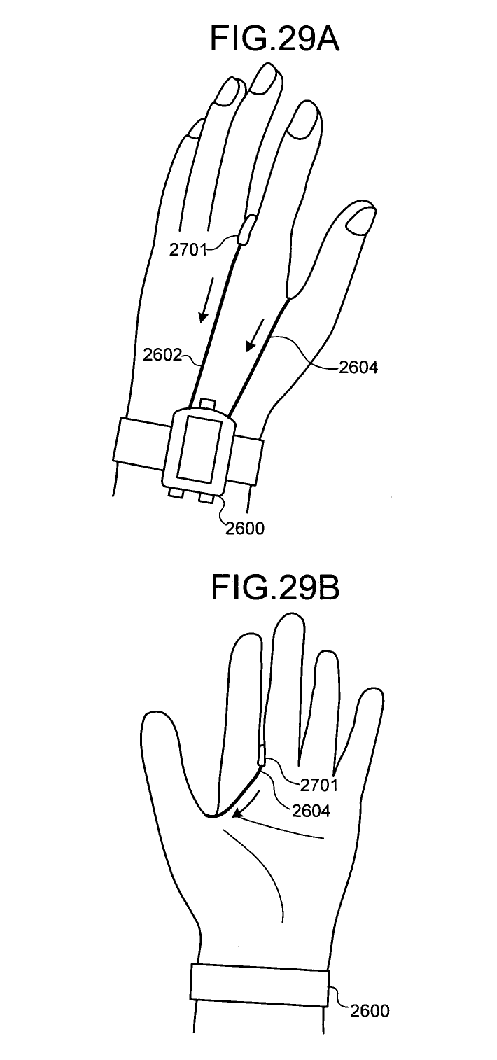

FIG. 29A and FIG. 29B show a state that the examinee wears the above-described bio-information processor 2600. FIG. 29A shows the back side of the hand, and FIG. 29B shows the palm side. As shown in these drawings, by pulling a sensor head 2701 from both of the back of the hand and the palm with a predetermined tension, it is possible to have stable attachment of the sensor head 2701. In the meantime, an attaching method is not limited to the methods shown in FIG. 29A and FIG. 29B and the sensor head 2701 may be attached between any fingers. In addition, if the sensor head 2701 can be fixed, the cables 2602 and 2604 may pass through a surface of any portions.

The bio-information measuring apparatus according to the first embodiment is provided with a cable winder in the bio-information processor, however, according to the bio-information measuring apparatus of a second embodiment, the cable winder is not needed by forming the cable for electrically connecting the sensor head to the bio-information processor by a raw material having elastic and the signal line.

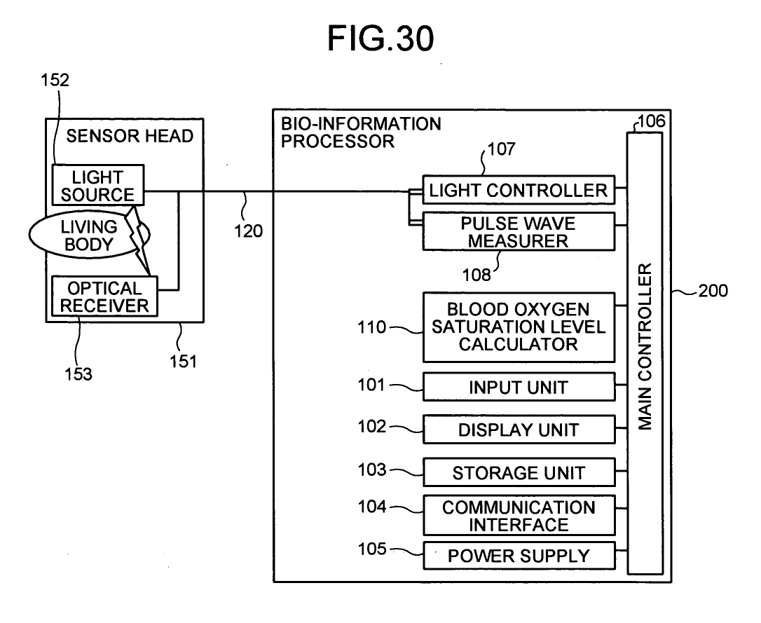

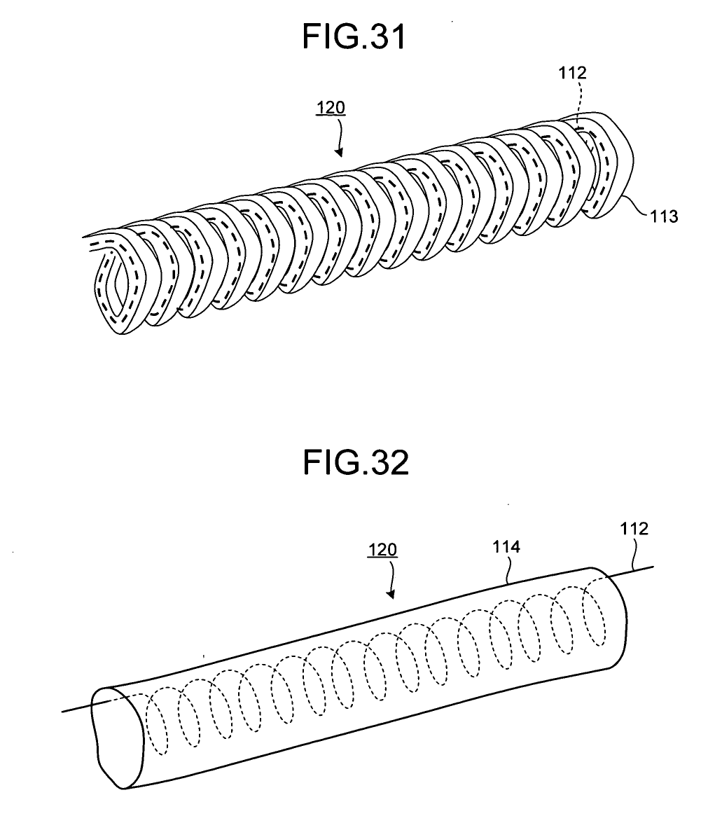

FIG. 30 is a block diagram of the bio-information measuring apparatus according to the second embodiment. In FIG. 30, the same reference numerals are given to the portions same as FIG. 1 and their explanations are herein omitted. A bio-information processor 200 shown in FIG. 30 is not provided with the cable winder, and a sensor head 151 is directly connected to the light source controller 107 and the pulse wave measurer 108 of the bio-information processor 200.

A cable 120 is located elastically in a longitudinal direction and includes a signal line therein. FIG. 31 shows an example of the cable 120. In FIG. 31, a signal line 112 is sealed in the tube 113 of a helix structure like a cable of a telephone receiver. According to this example, particularly, the tube 113 is preferably formed by a material with a higher hardness than that of a normal receiver cable in order to secure the tension.

FIG. 32 shows other example of the cable 120. In FIG. 32, the signal line 112 is embedded in spirals in a rubber material 114 with elasticity. Depending on such a structure as shown in these two examples, it is possible to provide the cable 120 with elasticity.

Thus, according to the bio-information measuring apparatus of the second embodiment, since a mechanism for winding the cable 112 is not needed, as compared to the bio-information measuring apparatus according to the first embodiment the same function can be realized by a more simple structure.

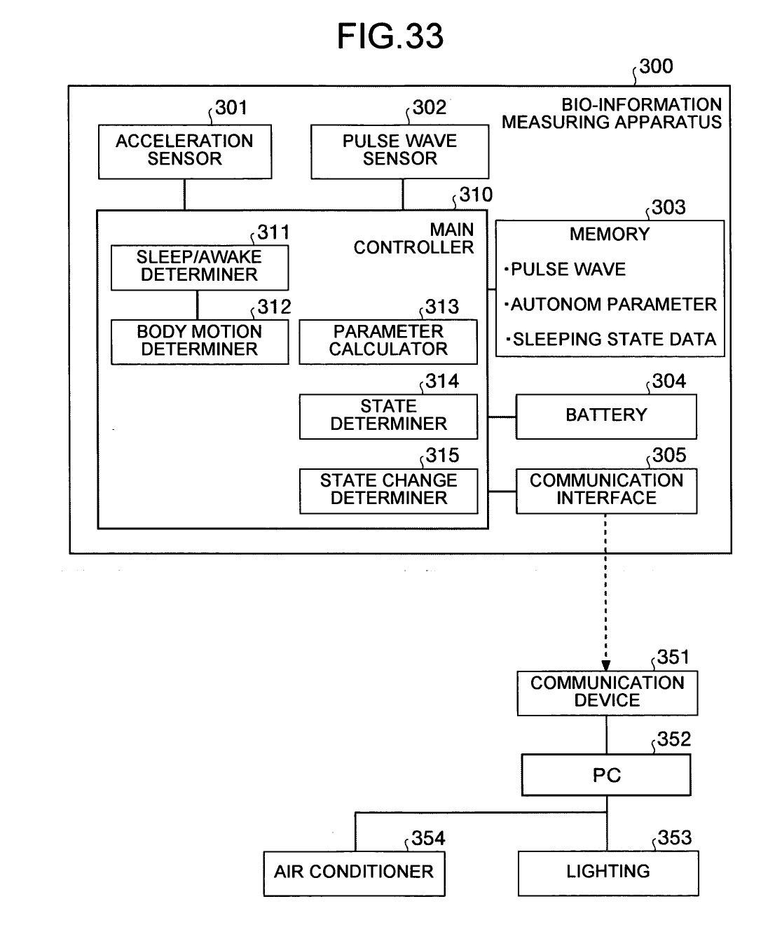

FIG. 33 is a block diagram a bio-information measuring apparatus 300 according to a third embodiment of the present invention, a communication device 351 that receives the data transmitted from the bio-information measuring apparatus 300, a lighting 353 that is controlled via a personal computer (PC) 352, and an air conditioner 354. As shown in FIG. 33, the bio-information measuring apparatus 300 according to the third embodiment is configured by an acceleration sensor 301, a pulse wave sensor 302, a memory 303, a battery 304, a communication interface 305, and a main controller 310. In the meantime, it is preferable that the bio-information measuring apparatus 300 is worn by the user so as not to interrupt a life environment of the user. For example, an integrally shaped ring type or the like including the acceleration sensor 301 and the pulse wave sensor 302 is conceivable, however, there is no limitation in its shape and portability.

The pulse wave sensor 302 is a pulse wave sensor of a reflection type and is configured by a red diode and a photo diode and the pulse wave sensor 302 serves to convert amount of blood from the reflection of a red diode emission into an electric signal in the photo diode. The pulse wave sensor 302 is needed to be attached to a portion where the pulse wave can be measured, for example, a tip of a finger of the user. However, according to the third embodiment, a portion where the pulse wave sensor 302 is attached is not limited to the tip of the finger of the user.

The acceleration sensor 301 is means that is worn by the user to measure the body motion, and for example, the acceleration sensor 301 is a three-axes acceleration sensor. In the meantime, the acceleration sensor 301 may be provided separately from the bio-information measuring apparatus 300 because kinds and accuracy of the obtained information are changed depending on a selected portion where the user wears the acceleration sensor 301.

The battery 304 serves to supply a power to the bio-information measuring apparatus 300. The power is supplied from the battery 304, so that the bio-information measuring apparatus 300 can calculate an autonomic parameter and can transmit the information about the autonomic parameter or the sleeping state data. In the meantime, as far as the battery 304 can be incorporated in the bio-information measuring apparatus 300, the shape or the like of the battery 304 is not limited.

On the basis of the data obtained from the pulse wave sensor 302 or the acceleration sensor 301, the main controller 310 may calculate the autonomic parameter of the user, may determine if the state is an awakening state or a sleeping state, and further, may specify the sleeping state data indicating a depth of sleeping if it is the sleeping state. This main controller 310 is configured by a sleep/awake determiner 311, a body motion determiner 312, a parameter calculator 313, a state determiner 314, and a state change determiner 315. Note that the depth of sleeping has the meaning of type of Non-REM or REM sleeping and the extent of the depth of Non-REM sleeping.

The sleep/awake determiner 311 is a unit that determines sleeping, and by the acceleration obtained from the acceleration sensor 301, it is detected if the user stops his or her motion continuously to determine if the user is awakening or sleeping. According to the third embodiment, on the basis of the data obtained from the acceleration sensor 301, the acceleration is calculated. For example, the determination level of the motion, Mg is calculated by the following mathematical Expression (1). In this case, it is assumed that an acceleration that was recorded previously is defined as (Xₚᵣₑ, Yₚᵣₑ, Zₚᵣₑ) and an acceleration that is measured currently is defined as (X_{cur}, Y_{cur}, Z_{cur}).
Mg=\sqrt{(Xₚᵣₑ−X_{cur})²+(Yₚᵣₑ−Y_{cur})²+(Zₚᵣₑ−Z_{cur})²}/256 (1)

Based on Expression (1), from the previously recorded acceleration (Xₚᵣₑ, Yₚᵣₑ, Zₚᵣₑ), obtaining a difference in the currently measured acceleration (X_{cur}, Y_{cur}, Z_{cur}) for each axis, Mg is calculated from a square of the sum of the square of each. Depending on if this calculated value is not less than 1 G (gal) or not, with or without of the body motion is determined, and further, in the case that the number of times not less than 1 G of the calculated value is not less than three times within five seconds, it is determined that the user is awakening, and in the case that the number of times not less than 1 G of the calculated value is less than three times within five seconds, it is determined that the user is sleeping. A first condition is that the number of times not less than 1 G of the calculated value is three times within five seconds. When it is measured that the calculated value is continuously not less than 1 G more than three times within five seconds for five minutes and more, even if the calculated value is not less than 1 G less than three times after five seconds, it is determined that the user is awakening only during a predetermined period of time. In the meantime, the optimum value is determined by the real measurement during this predetermined period of time.

The body motion determiner 312 may further determine if the body of the user is moved upon awakening by the acceleration obtained from the acceleration sensor 301 when the sleep/awake determiner 311 determines that the user is awakening. Specifically, even if the body motion is less than three times within five seconds, the above-described sleep/awake determiner 311 determines that the user is not moving when there is no body motion from the acceleration sensor 301 during twenty seconds in a predetermined period of time in which the user is determined to be awakening. Then, the sleep/awake determiner 311 may output this to the communication interface 305, and the communication interface 305 may transmit the autonomic parameter that is calculate by the parameter calculator 313.

The parameter calculator 313 is means that generates the nerve activation information, and specifically, the parameter calculator 313 may calculate the autonomic parameter for each predetermined time on the basis of the pulse waves that are calculated by the pulse wave sensor 302 and the pulse waves that are accumulated in the memory 303 and may accumulate the calculated autonomic parameters in the memory 303. According to a specific calculating method, replacing variation of a cardiac beat obtained from the pulse wave data accumulated in the memory 303 with the pulse wave, a frequency analysis is carried out, and a ratio with respect to an entire power value of a power value of a component about 0.3 Hz in a power spectrum resulted from this is calculated as HF and a value obtained by dividing the power value of the component about 0.1 Hz in the power spectrum by the power value of a component about 0.3 Hz is calculated as LF In addition, HF is a value to reflect the activation state of a parasympathetic nerve of an autonomic nerve system and LF is a value to reflect the activation state of a sympathetic nerve of the autonomic nerve system; and these HL and LF are determined to be an autonomic parameter. In the meantime, the autonomic parameter is not limited to the above-described values of HL and LF and the autonomic parameter may be a value that can be determined on the basis of the sleeping state data or a value representing the autonomic nerve state necessary to control the home electric appliance or the like. Further, with respect to a predetermined period of time during which the autonomic parameter is calculated, the optimum one is determined by the real measurement.

The state determiner 314 may specify the sleeping state data indicating the sleeping data of the user on the basis of the autonomic parameters accumulated in the memory 303 and may accumulate it in the memory 303. According to the third embodiment, the state determiner 314 may specify if the sleeping is Non-REM sleeping or REM sleeping depending on if HF is larger than a first predetermined value or not. Then, when specifying it as the Non-REM sleeping, further, the state determiner 314 may specify it as a deep sleeping state or a shallow sleeping state depending on if HF is larger than a second predetermined value or not. In addition, by defining a predetermined value for specifying the sleeping state data with respect to the value of LF in the same way and determining the sleeping state data by the values of HF and LF, it is possible to specify the sleeping state data with a high degree of accuracy. Further, it is necessary to set the optimum value by the real measurement because the above-described predetermined value varies from person to person. In the meantime, a method to specify the sleeping state data is not limited to the above-described method and it may be a method whereby the sleeping state data can be specified on the basis of the autonomic parameter calculated from the parameter calculator 313. Further, according to the third embodiment, the sleeping state data is divided into three sates, namely, REM, the shallow sleeping state, and the deep sleeping state, however, not limited to this division, the sleeping state data may be divided only into REM and Non REM or into more sleeping sates.

The state change determiner 315 is equivalent to the state change determining means of the present invention, and the state change determiner 315 may determine if the sleeping state data is changed on the basis of the sleeping state data inputted from the state determiner 314 and the sleeping state data previously specified and accumulated in the memory 303. If the sleeping state data is determined to be chanted, the state change determiner 315 may transmit this to the communication interface 305 and may transmit the sleeping state data from the communication interface 305.

The communication interface 305 may transmit the calculated autonomic parameter or the specified sleeping state data.

The memory 303 may accumulate the pulse wave, the autonomic parameter, and the sleeping state data therein. Specifically, the pulse wave, the autonomic parameter, and the sleeping state data are needed in order to calculate the autonomic parameter, to specify the sleeping state data or transmit the sleeping state data from the communication interface 104, and to determine if the sleeping sate data is changed as compared to the calculated sleeping state data, respectively. Further, the information to be accumulated in the memory 303 is not limited to the pulse data, the autonomic parameter, and the sleeping state data, and it may be the information needed to calculate the autonomic parameter or to transmit the autonomic parameter.

The structure of the bio-information measuring apparatus 300 according to the third embodiment of the present invention is as described above. Then, the communication device 351 that receives the information transmitted from the bio-information measuring apparatus 300, the PC 352 that is an external device that obtains the information and controls the home electric appliance or the like, the lighting 353 to be controlled by the PC, and the air conditioner 354 will be described below.

The communication device 351 may receive the autonomic parameter or the sleeping state data transmitted from the communication interface 305 of the bio-information measuring apparatus 300 and may output the received autonomic parameter or the sleeping state data to the PC 352. As a communication system with the communication interface 305 of the bio-information measuring apparatus 300, a communication system capable of being used in a life environment of the user is available, and for example, a communication system using a wireless transmission or the like is conceivable.

The PC 352 may control the lighting 353 or the air conditioner 354 on the basis of the autonomic parameter or the sleeping state data that are inputted from the communication device 351 in order to make the environment suitable for the user. For example, as a result of calculation executed by the PC 352 on the basis of HF and LF as the inputted autonomic parameter, if the user is determined to be excited, a color of the lighting 353 is changed into a warm color in order to relax the user Alternatively, control to decrease brightness or the like is conceivable. Further, when the sleeping state is determined to be deep from the sleeping state data, control to adjust a temperature of the air conditioner 354 is conceivable. Since the lighting 353 and the air conditioner 354 are controlled due to the bio-information to be transmitted, it is possible to provide a life environment comfortable for the user. In addition, only when the sleeping state data is changed or only when the user is not moving upon awakening, the information is transmitted, so that the processing by the PC 352 can be decreased. In the meantime, the home electric appliance to be controlled by the PC 352 is not limited to the lighting 353 and the air conditioner 354.

The lighting 353 and the air conditioner 354 are controlled by the PC 352 in order to provide an environment comfortable for the user. Therefore, it is necessary that the lighting 353 and the air conditioner 354 can communicate to the PC 352, however, there is no specific limitation with respect to a communication system.

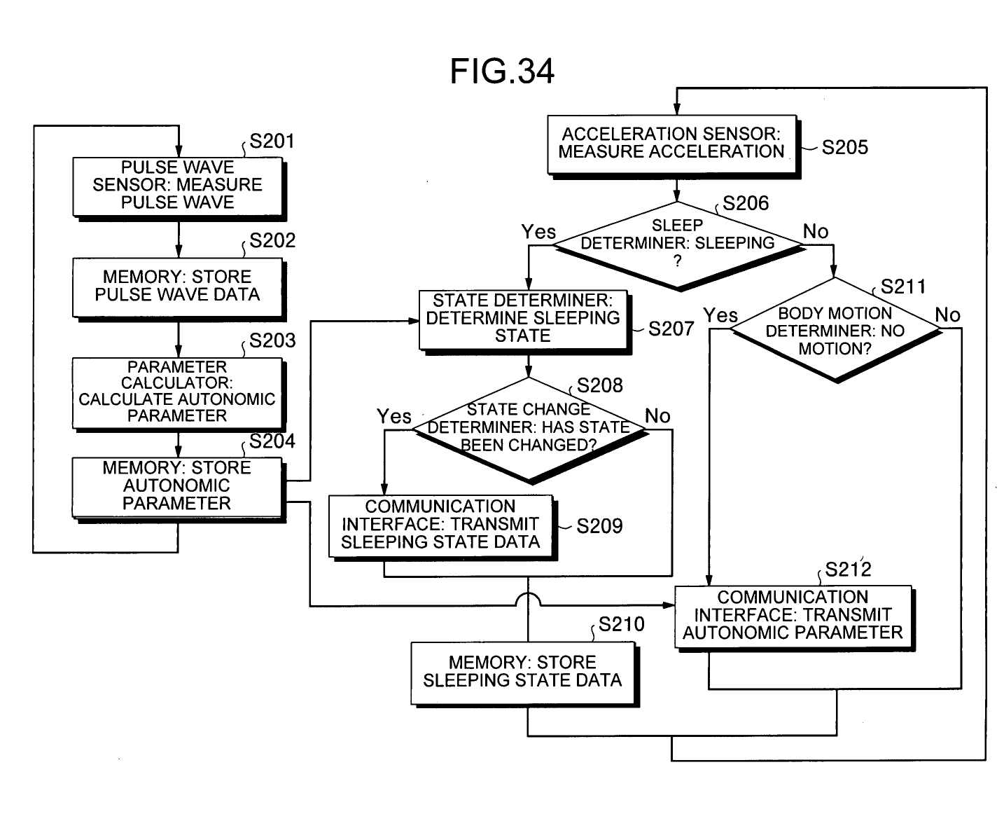

In the next place, the processing of the bio-information measuring apparatus 300 according to the present embodiment that is configured as described above will be described below. FIG. 34 is a flowchart that depicts an example of a procedure of the processing of the bio-information measuring apparatus 300 according to the third embodiment. In the meantime, the processing of the bio-information measuring apparatus 300 is not limited to the following procedure.

At first, the pulse wave sensor 302 may measure the pulse wave (step S201). The measured pulse wave is accumulated in the memory 303 (step S202). On the basis of this measure or accumulated pulse wave, the parameter calculator 313 may calculate the autonomic parameter (step S203). Specifically, the autonomic parameter is an activation value HF reflecting the activation state of the parasympathetic nerve of the autonomic nerve system and the activation value LF reflects the activation state of the sympathetic nerve of the autonomic nerve system. Then, the calculated autonomic parameter is accumulated in the memory 303 (step S204).

The acceleration sensor 301 may measure the body motion of the user by measuring the acceleration (step S205). Then, the sleep/awake determiner 311 may determine if the user is awakening or sleeping on the basis of the body motion that is detected by the acceleration sensor 301 (step S206). Specifically, if the acceleration not less than 1 G is detected three times and more within five seconds in the acceleration measured by the acceleration sensor 301, the sleep/awake determiner 311 may determine that the user is awakening, and if the acceleration not less than 1 G is detected less than three times within five seconds, the sleep/awake determiner 311 may determine that the user is sleeping. In addition, in the case that the acceleration not less than 1 G is detected three times and more within five seconds continuously for five minutes and more, even if the calculated value is not less than 1 G less than three times after five seconds, the sleep/awake determiner 311 may determine that the user is awakening.

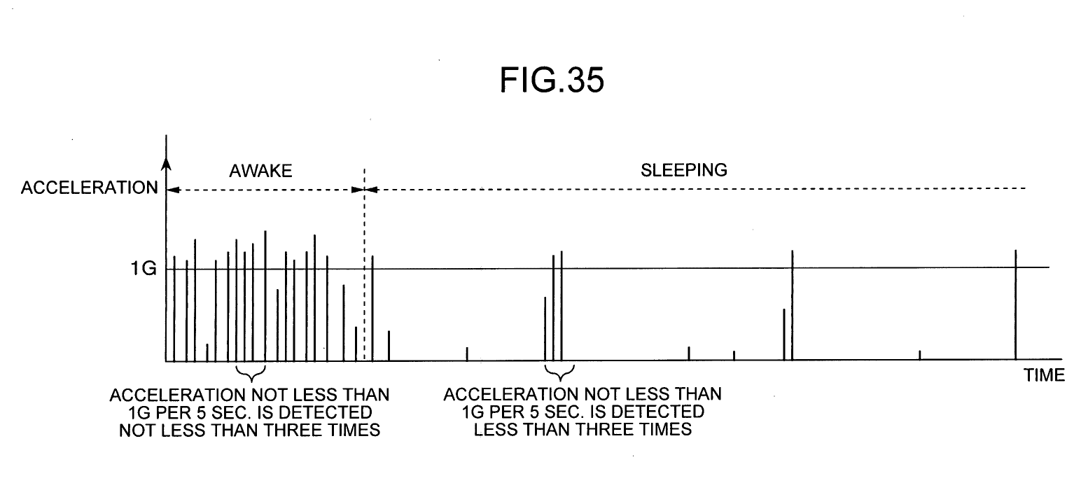

FIG. 35 is a frequency of the body motion when the user is awakening and sleeping by an acceleration that is detected by the acceleration sensor 301. As shown in FIG. 35, if the acceleration not less than 1 G is detected three times and more within five seconds, the sleep/awake determiner 311 may determine that the user is awakening and if the acceleration not less than 1 G is detected less than three times within five seconds, the sleep/awake determiner 311 may determine that the user is sleeping.

Returning to FIG. 34, when the sleep/awake determiner 311 determines that the user is sleeping (step S206: Yes), the state determiner 314 may obtain the autonomic parameter accumulated in the memory 303 and may specify the sleeping state data (step S207). In detail, on the basis of HF and LF determined as the autonomic parameter, the state determiner 314 may specify the sleeping state data. The sleeping state data is divided into three states, namely, the REM sleeping state, the shallow sleeping state, and the deep sleeping state. Then, as comparing the sleeping state data that is specified currently to the sleeping state data that was previously specified accumulated in the memory 303 by the state change determiner 315, it is determined if the sleeping state data is changed or not (step S208). Specifically, if the previous sleeping state data is shallow, when the currently specified sleeping state data is the deep sleeping state or the REM sleeping, it is determined that the sleeping state has been changed. Then, when the sleeping state data is determined to be changed by the state change determiner 315 (step S208: YES), the communication interface 305 may transmit the sleeping state data that is currently specified (step S209). In addition, when it is determined that the sleeping state data is not changed by the state change determiner 315 (step S208: No), the processing is not carried out particularly. After that, the specified sleeping state data is accumulated in the memory 303 (step S210). In the meantime, the accumulated sleeping state data is used to determine if the sleeping state data of the user is changed next time.

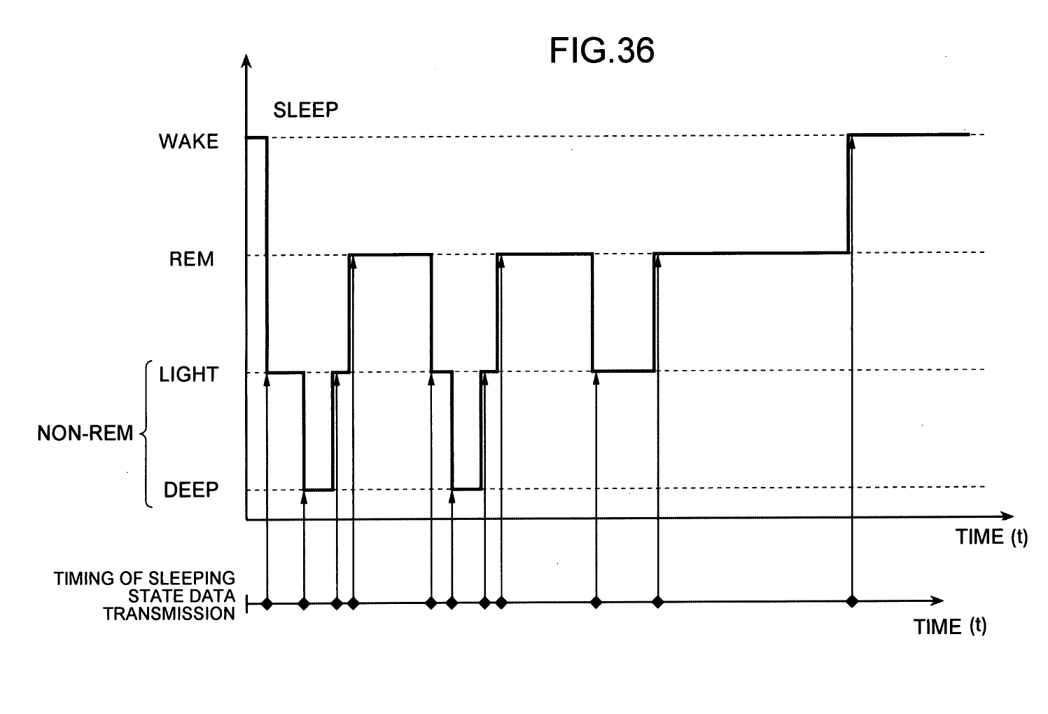

FIG. 36 is a data transmitting time of the sleeping state data by change of the sleeping state of the user. In FIG. 36, WAKE shows a state that the user is awakening, and as described above, a case that the acceleration detected by the acceleration sensor 301 not less than 1 G is detected three times and more within five seconds may be equivalent to WAKE. The REM, the shallow sleeping state, and the deep sleeping state are the sleeping state data determined by the state change determiner 315. Then, at a time represented by a root of an arrow just after the sleeping state data is changed, the sleeping state data is transmitted to the communication interface 305. In addition, the sleeping state data to be transmitted is the sleeping state after changing represented by an arrow.

Returning to FIG. 34, when the sleep/awake determiner 311 determines that the user is awakening (step S206: No), the body motion determiner 312 may determine if the user is moving or not (step S211). Specifically, when the acceleration value obtained by the acceleration sensor 301 not less than 1 G is not measured for twenty seconds, the sleep/awake determiner 311 determines that the user is not moving. In addition, a case that the acceleration value obtained by the acceleration sensor 301 not less than 1 G is not measured for twenty seconds is defined as a second condition. If the body motion determiner 312 determines that the user is not moving (step S211: Yes), the communication interface 305 may transmit the autonomic parameter accumulated in the memory 303 (step S212). If the body motion determiner 312 determines that the user is moving (step S211: No), the processing is not carried out particularly. The autonomic parameter in a predetermined time period may be displayed when the calculation of the autonomic parameter within the time period results in no motion.

According to the third embodiment, when the user is sleeping, the sleeping state data is transmitted, and when the user is awakening, the autonomic parameter is transmitted. In other words, a different parameter is transmitted when sleeping and when awakening, so that it is possible to control the home electric appliance suitable for the state of the user. In the meantime, the processing procedure from measurement of the pulse wave to accumulation of the autonomic parameter from the step S201 to the step S204 and the processing procedure from measurement of acceleration to transmission of the sleeping state data or the autonomic parameter and accumulation of the sleeping state data from the step S205 to the step S212 are carried out in parallel, and timing such as measurement of the pulse wave and measurement of the acceleration or the like is not limited.

In addition, determining if change of the sleeping state data of the user by the state change determiner 315, only when the current sleeping state data is different from the previous sleeping state data, the number of communication can be decreased and power saving can be made by transmitting the sleeping state data by the communication interface 305. Further, only when the body motion determiner 312 determines that the user is not moving, the data communication is carried out, so that the number of communication can be decreased and power saving can be made. In addition, since the body motion determiner 312 calculates the autonomic parameter on the basis of the pulse wave when the user is not moving and transmits the calculated autonomic parameter, the data having few influences such as a noise and having a high reliability can be transmitted.

In the meantime, a value of a parameter described in the third embodiment is indicated only as one example, and the present embodiment is not limited to these values. Specifically, determination by the sleep/awake determiner 311 is not limited to if the value measured by the acceleration sensor 301 is not less than 1 G or not, and the number of times for determining if the user is sleeping or awakening is not limited to three times for five seconds. A time for determining that the user is awakening by the body motion determiner 312 is not limited to five minutes, and a time for determining that the user is not moving is not limited to twenty seconds.

According to the bio-information measuring apparatus according to the third embodiment, when the sleeping state data is changed when the user is sleeping, the sleeping state data is transmitted, however, according to the bio-information measuring apparatus according to a fourth embodiment, when the body motion of the user is measured by the acceleration sensor 301 when the user is sleeping, the sleeping state data is transmitted.

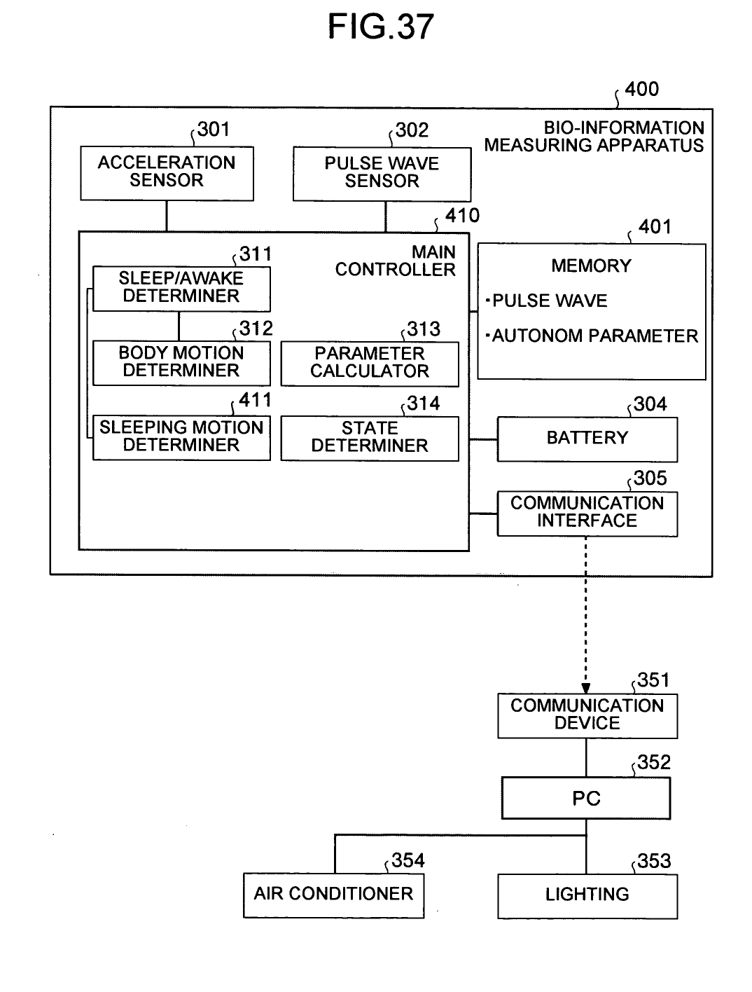

FIG. 37 is a block diagram of a bio-information measuring apparatus 400 according to the fourth embodiment As shown in FIG. 37, the bio-information measuring apparatus 400 according to the fourth embodiment is configured by the acceleration sensor 301, the pulse wave sensor 302, a memory 401, the battery 304, the communication interface 305, and a main controller 410; and the main controller 310 according to the third embodiment is changed to the main controller 410 of which processing is different from that of the main controller 310 and the memory 303 is changed to the memory 401 of which accumulated information is different from that of the memory 303. In the following explanation, the same reference numerals are given to the same constituent elements as the above-described third embodiment and their explanations are herein omitted.

The main controller 410 is configured by the sleep/awake determiner 311, the body motion determiner 312, the parameter calculator 313, the state determiner 314, and a sleeping body motion determiner 411. In the main controller 410, the state change determiner 315 is removed from the bio-information measuring apparatus 300 and in place of it, the sleeping body motion determiner 411 is added.

The sleeping body motion determiner 411 may determine with or without of the body motion when the user is sleeping in the case that the sleep/awake determiner 311 determines that the user is sleeping. In other words, since it is known that the body motion occurs when the sleeping state is changed, assuming that the sleeping state is changed when determining that there is the body motion, the sleeping state data is transmitted from the communication interface 305.

The memory 401 accumulates the pulse wave and the nerve activation parameter and does not accumulate the sleeping state data accumulated in the memory 303 according to the first embodiment. In other words, according to the fourth embodiment, change of the sleeping state data is not determined. As a matter of course, the information to be accumulated in the memory 401 is not limited to the pulse wave and the autonomic parameter, and the information necessary to calculate the autonomic parameter or to transmit it is available as this information.

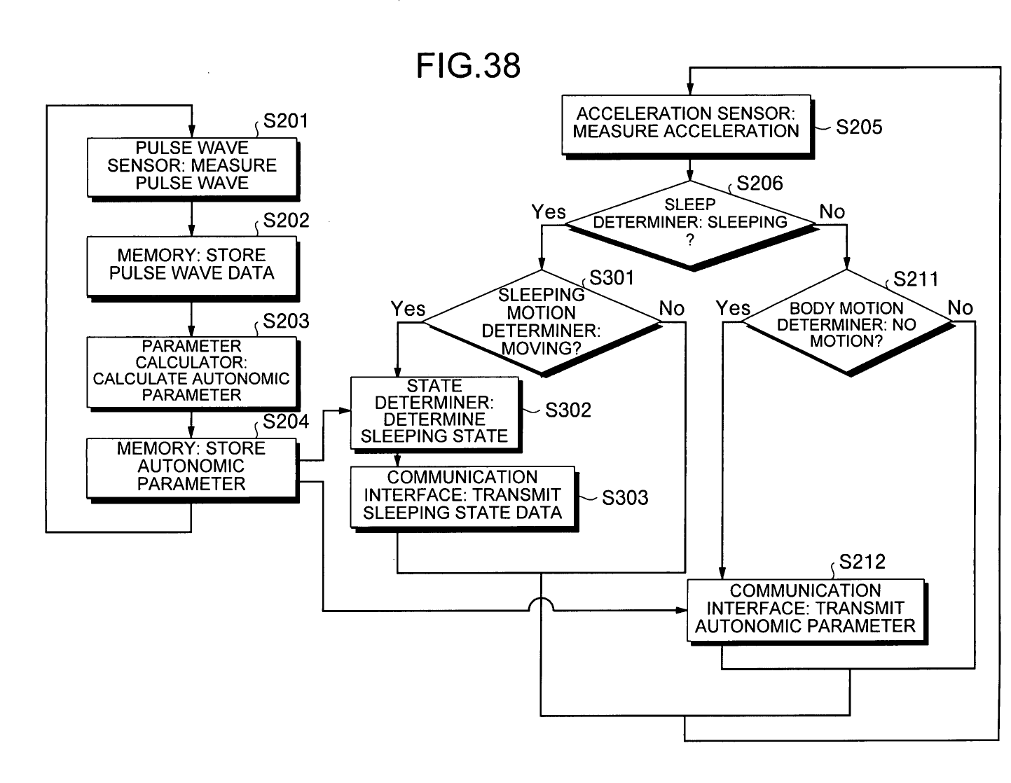

In the next place, the processing of the bio-information measuring apparatus 400 according to the present embodiment that is configured as described above will be described below. FIG. 38 is a flowchart of processing in the bio-information measuring apparatus 400 according to the fourth embodiment. In the meantime, the processing of the bio-information measuring apparatus 400 is not limited to the following procedure.

At first, as same as the steps from S201 to S204 shown in FIG. 34 according to the third embodiment, calculating the autonomic parameter from the pulse wave, the autonomic parameter is accumulated in the memory 401.

Calculating the acceleration as same as the step S205 and the step S206 shown in FIG. 34 according to the third embodiment, the sleep/awake determiner 311 determines if the user is awakening or not on the basis of the calculated acceleration. Then, when the sleep/awake determiner 311 determines that the user is sleeping (step S206: Yes), the sleeping body motion determiner 411 may determine with or without of the body motion (step S301). In determination of with or without of the body motion by the sleeping body motion determiner 411, for example, the sleeping body motion determiner 411 determines that there is the body motion, for example, when the acceleration sensor 301 detects a value not less than 1 G two times for ten seconds. This condition that the acceleration not less than 1 G is detected two times for ten seconds is equivalent to a third condition. However, in determination of with or without of the body motion, the number of times is not limited to two times because there is an individual difference due to a difference of the user and it is necessary to set the optimum value for each user by the real measurement.

When the sleeping body motion determiner 411 determines that there is the body motion (step S301: Yes), the state determiner 314 may specify the sleeping state data (step S302). It is assumed that a method to specify the sleeping state data is the same as the step S207 of the first embodiment. The communication interface 305 may transmit the sleeping state data specified by the state determiner 314 (step S303).

When the sleep/awake determiner 311 determines that the user is awakening (step S206: No), as same as the step S211 and the step S212 shown in FIG. 34 according to the third embodiment, only when the user is not moving, the communication interface 305 may transmit the autonomic parameter.

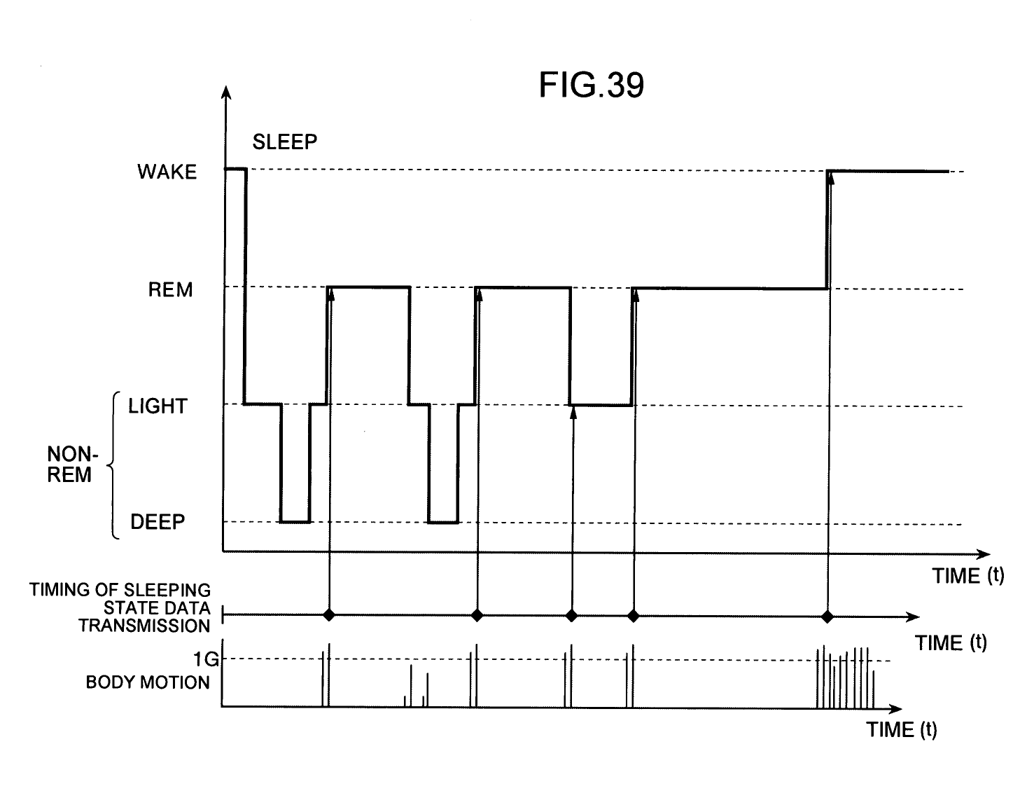

FIG. 39 is a data transmitting time of the sleeping state data by measuring of the movement of the user. When the sleeping body motion determiner 411 determines that there is the body motion, the communication interface 305 may transmit the sleeping state data at that time. In addition, the communication interface 305 may transmit the sleeping state data represented by a point of an arrow shown in FIG. 39 as the sleeping state data at a time represented by a root of the arrow.

According to the fourth embodiment, only when there is the motion when the user is sleeping, the number of communication can be decreased and power saving can be made by transmitting the sleeping state data.

According to the fourth embodiment, as same as the third embodiment, only when the body motion determiner 312 determines that the user is not moving, the data communication is carried out, so that the number of communication can be decreased and power saving can be made. In addition, since the body motion determiner 312 calculates the autonomic parameter on the basis of the pulse wave when the user is not moving and transmits the calculated autonomic parameter, the data having few influences such as a noise and having a high reliability can be transmitted. Further, also according to the fourth embodiment, as same as the third embodiment, the described values of the parameter are indicated only as an example, and the present embodiment is not limited to these values.

In the meantime, according to the fourth embodiment, when it is determined that there is the motion when the user is sleeping, the communication interface 305 transmits the sleeping state data at that time, however, the determination is not limited to if there is the motion when the user is sleeping or not, and for example, it may be decided if the data communication should be carried out or not by combining the above determination with the determination if the sleeping state data is changed or not during sleeping in the third embodiment.

In the meantime, the present invention is not limited to the above-described embodiments as they are, and in an execution phase, a modification of a constituent element will become possible without departing from the scope thereof. In addition, by an appropriate combination of plural constituent elements disclosed in the above-described embodiment and modification, various inventions can be made. For example, some constituent elements may be deleted from among all constituent elements that are disclosed in the embodiments and the modifications. Further, the constituent elements of different modifications may be appropriately combined.

For example, the pulse wave sensor 302 and the acceleration sensor 301 that are explained in the third and fourth embodiments can be replaced with the structure configured by the sensor head 151, the cable 111, and the cable winder 109 that is explained in the first embodiment. In the same way, the pulse wave sensor 302 and the acceleration sensor 301 that are explained in the third and fourth embodiments can be replaced with the structure configured by the sensor head 151 and the cable 120 that is explained in the first embodiment

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A bio-information measuring apparatus, comprising:
a detector including a light source emitting light to a hand of an examinee and an optical receiver receiving light transmitted through the hand from the light source;
a supporter bent to clamp a paddle between fingers of the examinee, a clamping portion of the supporter being provided with the detector;
a measurer worn on the examinee, measuring bio-information based on pulse wave data obtained from the optical receiver; and
a puller pulling the support member toward the measurer.

2. The bio-information measuring apparatus according to claim 1, wherein the supporter is made of a resin that is formed in a bent shape along the paddle.

3. The bio-information measuring apparatus according to claim 1, wherein the puller includes a cable connecting the supporter to the measurer, and a winder winding the cable.

4. The bio-information measuring apparatus according to claim 3, wherein the winder is disposed in the measurer.

5. The bio-information measuring apparatus according to claim 3, wherein the winder is disposed in an end portion of the clamping portion.

6. The bio-information measuring apparatus according to claim 3, wherein the cable includes a first signal line where a signal for driving the light source is transmitted from the measurer, and a second signal line where a signal received by the optical receiver is transmitted to the measurer.

7. The bio-information measuring apparatus according to claim 1, wherein the puller connects the supporter to the measurer and is elastic.

8. The bio-information measuring apparatus according to claim 3, wherein the puller connects the supporter to the measurer and is elastic.

9. The bio-information measuring apparatus according to claim 8, wherein the cable includes a first signal line where a signal for driving the light source is transmitted from the measurer, and a second signal line where a signal received by the optical receiver is transmitted to the measurer.

10. The bio-information measuring apparatus according to claim 1, wherein the supporter has a cushion that is provided around at least one of the light source and the optical receiver, the cushion being in contact with the examinee.

11. The bio-information measuring apparatus according to claim 1, wherein the supporter has a cushion that wraps the light source and the optical receiver.

12. The bio-information measuring apparatus according to claim 1, wherein the clamping portion is formed to be made wider from the detector toward a wrist of the examinee.

13. The bio-information measuring apparatus according to claim 1, wherein the supporter has a first length from a top of a bent portion of the supporter to an end of the clamping portion at a back side of the hand, and a second length from the top of the bent portion to another end of the clamping portion at a palm side, the second length being different from the first length.

14. The bio-information measuring apparatus according to claim 1, wherein one of the light source and the optical receiver is disposed in the clamping portion at a back side of the hand, another is disposed in the clamping portion at a palm side.

15. The bio-information measuring apparatus according to claim 14, wherein the supporter has a first length from the optical receiver to an end of the clamping portion at a wrist side and a second length from the optical receiver to another end of the clamping portion at the wrist side, the second length being different from the first length.

16. The bio-information measuring apparatus according to claim 1, wherein the light source and the optical receiver are both disposed in the clamping portion at one of a back side and a palm side of the hand, the optical receiver receives light emitted from the light source and then reflected in the hand.

17. The bio-information measuring apparatus according to claim 1, wherein the supporter includes a rotating member rotating along the paddle at a position where the detector is provided.

18. A bio-information measuring apparatus comprising:
a parameter calculator calculating parameter indicating a state of the autonomic nerve activation based on a pulse wave;
a body motion measurer measuring body motion information indicating a body motion of an examinee;
a sleep/awake determiner determining whether the examinee is awakening or sleeping based on the body motion information;
a body motion determiner determining whether the examinee is moving based on the body motion information when the sleep/awake determiner determines that the examinee is awakening;
a first communication interface transmits the parameter calculated by the parameter calculator to an external device via a network when the body motion determiner determines that the examinee is not moving;
a sleeping state determiner determining sleeping state information indicating a depth of sleeping from the parameter calculated by the parameter calculator when the sleep state determiner determines that the examinee is sleeping;
a state change determiner determines whether the sleeping state information is changed compared with sleeping state information that has been determined by the sleeping state determiner; and
a second communication interface transmits the sleeping state information to the external device via the network when the state change determiner determines that the sleeping state information is changed.

19. The bio-information measuring apparatus according to claim 18, wherein the sleep state determiner determines that the examinee is awakening when the body motion information satisfies a first condition, and determines that the examinee is sleeping when the body motion information does not satisfy the first condition.

20. The bio-information measuring apparatus according to claim 18, wherein the body motion determiner determines that the examinee is moving when the body motion information satisfies a second condition, and determines that the examinee is not moving when the body motion information does not satisfy the second condition.

21. A bio-information measuring apparatus comprising:
a parameter calculator calculating parameter indicating a state of the autonomic nerve activation based on a pulse wave;
a body motion measurer measuring body motion information indicating a body motion of an examinee;
a sleep/awake determiner determining whether the examinee is awakening or sleeping based on the body motion information;
a body motion determiner determining whether the examinee is moving based on the body motion information when the sleep/awake determiner determines that the examinee is awakening;
a first communication interface transmits the parameter calculated by the parameter calculator to an external device via a network when the body motion determiner determines that the examinee is not moving;
a sleeping body motion determiner determining whether the examinee is moving in sleep based on the body motion information when the sleep/awake determiner determines that the examinee is sleeping;
a sleeping state determiner determining sleeping state information indicating a depth of sleeping from the parameter calculated by the parameter calculator when the body motion determiner determines that the examinee is moving; and
a second communication interface transmits the sleeping state information to the external device via the network.

22. The bio-information measuring apparatus according to claim 21, wherein the sleep state determiner determines that the examinee is awakening when the body motion information satisfies a first condition, and determines that the examinee is sleeping when the body motion information does not satisfy the first condition.

23. The bio-information measuring apparatus according to claim 21, wherein the body motion determiner determines that the examinee is moving when the body motion information satisfies a second condition, and determines that the examinee is not moving when the body motion information does not satisfy the second condition.

24. The bio-information measuring apparatus according to claim 21, wherein the sleeping body motion determiner determines that the examinee is moving in sleep when the body motion information satisfies a third condition, and determines that the examinee is not moving in sleep when the body motion information does not satisfy the third condition.

25. A bio-information measuring apparatus, comprising:
a detector including a light source emitting light to a hand of an examinee and an optical receiver receiving light transmitted through the hand from the light source;
a supporter bent to clamp a paddle between fingers of the examinee, a clamping portion of the supporter being provided with the detector;
a measurer worn on the examinee, measuring bio-information based on pulse wave data obtained from the optical receiver; and
a puller pulling the support member toward the measurer,
wherein the measurer includes
a parameter calculator calculating parameter indicating a state of the autonomic nerve activation based on the pulse wave data;
a body motion measurer measuring body motion information indicating a body motion of the examinee;
a sleep/awake determiner determining whether the examinee is awakening or sleeping based on the body motion information;
a body motion determiner determining whether the examinee is moving based on the body motion information when the sleep/awake determiner determines that the examinee is awakening;
a first communication interface transmits the parameter calculated by the parameter calculator to an external device via a network when the body motion determiner determines that the examinee is not moving;
a sleeping state determiner determining sleeping state information indicating a depth of sleeping from the parameter calculated by the parameter calculator when the sleep state determiner determines that the examinee is sleeping;
a state change determiner determines whether the sleeping state information is changed compared with sleeping state information that has been determined by the sleeping state determiner; and
a second communication interface transmits the sleeping state information to the external device via the network when the state change determiner determines that the sleeping state information is changed.

26. A bio-information measuring apparatus, comprising:
a detector including a light source emitting light to a hand of an examinee and an optical receiver receiving light transmitted through the hand from the light source;
a supporter bent to clamp a paddle between fingers of the examinee, a clamping portion of the supporter being provided with the detector,
a measurer worn on the examinee, measuring bio-information based on pulse wave data obtained from the optical receiver; and
a puller pulling the support member toward the measurer,
wherein the measurer includes
a parameter calculator calculating parameter indicating a state of the autonomic nerve activation based on the pulse wave data;
a body motion measurer measuring body motion information indicating a body motion of the examinee;
a sleep/awake determiner determining whether the examinee is awakening or sleeping based on the body motion information;
a body motion determiner determining whether the examinee is moving based on the body motion information when the sleep/awake determiner determines that the examinee is awakening;
a first communication interface transmits the parameter calculated by the parameter calculator to an external device via a network when the body motion determiner determines that the examinee is not moving;
a sleeping body motion determiner determining whether the examinee is moving in sleep based on the body motion information when the sleep/awake determiner determines that the examinee is sleeping;
a sleeping state determiner determining sleeping state information indicating a depth of sleeping from the parameter calculated by the parameter calculator when the body motion determiner determines that the examinee is moving; and
a second communication interface transmits the sleeping state information to the external device via the network.

27. A bio-information measuring apparatus, comprising:
a detector including a light source emitting light to a hand of an examinee and an optical receiver receiving light transmitted through the hand from the light source;
a supporter bent to clamp a paddle between fingers of the examinee, a clamping portion of the supporter being provided with the detector;
a measurer worn on the examinee, measuring bio-information based on pulse wave data obtained from the optical receiver; and
a puller pulling the support member toward the measurer,
wherein the measurer includes
a parameter calculator calculating parameter indicating a state of the autonomic nerve activation based on the pulse wave data;
a body motion measurer measuring body motion information indicating a body motion of the examinee;
a sleep/awake determiner determining whether the examinee is awakening or sleeping based on the body motion information; and
a sleeping state determiner determining sleeping state information indicating a depth of sleeping from the parameter calculated by the parameter calculator when the sleep/awake determiner determines that the examinee is sleeping.
